SEQUENCE LISTING (32 sequences)

SEQ ID NO: 1 - DNA, 361 bp, synthetic construct
  misc_feature 1..361: note = Synthetic: alpha-BauA Heavy Chain
  source 1..361: mol_type = other DNA
gaggtgcagctgcaggagtctggggctgacctggtgaggcctgggtcctcagtgaagatttcctgcaaggcttctggctatgcattcagtaactactggatgcactggatgaagcagaggcctggacagggtcttgagtggattggacagatttatcctggagatggtgatactaactacaatggaaagttcaagggtaaagccacactgactgcagacaaatcctccagctcagcctacatgcagctcagcagcctaatatctgaggactctgcggtctatttctgtgcaagaggggatttttactacggtagcccgtttgcttactggggccaagggactctggtcactgtctctgcag

SEQ ID NO: 2 - protein, 120 aa, synthetic construct
  REGION 1..120: note = Synthetic: alpha-BauA Heavy Chain
EVQLQESGADLVRPGSSVKISCKASGYAFSNYWMHWMKQRPGQGLEWIGQIYPGDGDTNYNGKFKGKATLTADKSSSSAYMQLSSLISEDSAVYFCARGDFYYGSPFAYWGQGTLVTVSA

SEQ ID NO: 3 - protein, 8 aa, synthetic construct
  REGION 1..8: note = Synthetic: CDR1
GYAFSNYW

SEQ ID NO: 4 - protein, 8 aa, synthetic construct
  REGION 1..8: note = Synthetic: CDR2
IYPGDGDT

SEQ ID NO: 5 - protein, 13 aa, synthetic construct
  REGION 1..13: note = Synthetic: CDR3
ARGDFYYGSPFAY

SEQ ID NO: 6 - DNA, 337 bp, synthetic construct
  misc_feature 1..337: note = Synthetic: Light Chain
  source 1..337: mol_type = other DNA
gatattgtgatgactcaggctgcaccctctgtacctgtcactcctggagagtcagtatccatctcctgcaggtctagtaagagtctcctgcatagtaatggcaacacttacttgtattggttcctgcagaggccaggccagtctcctcagctcctgatttatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgctcacgttcggtgctgggaccaagctggagctgaaac

SEQ ID NO: 7 - protein, 112 aa, synthetic construct
  REGION 1..112: note = Synthetic: Light Chain
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPLTFGAGTKLELK

SEQ ID NO: 8 - protein, 11 aa, synthetic construct
  REGION 1..11: note = Synthetic: CDR1
KSLLHSNGNTY

SEQ ID NO: 9 - skipped

SEQ ID NO: 10 - protein, 9 aa, synthetic construct
  REGION 1..9: note = Synthetic: CDR3
MQHLEYPLT

SEQ ID NO: 11 - DNA, 346 bp, synthetic construct
  misc_feature 1..346: note = Synthetic: alpha-OmpW2 Heavy Chain
  source 1..346: mol_type = other DNA
gaggtgcagctgcaggagtctggggctgaacttgtgaggccaggggccttaattaagttgtcctgcaaagcttctggcttcaatgttaaagactactatatgcaatgggtgaagcagaggcctgaacagggcctggagtggattggatggattgatcctgagaatgggaaaactatatgtgacccgaagttccagggcaaggccagtataacagcagacacatcctccaacacagcctacctgcacctcagcagcctgacatctgaggacactgccgtctattactgtgctactacatggggggttccttactggggccaagggactctggtcactgtctccgcag

SEQ ID NO: 12 - protein, 115 aa, synthetic construct
  REGION 1..115: note = Synthetic: alpha-OmpW2 Heavy Chain
EVQLQESGAELVRPGALIKLSCKASGFNVKDYYMQWVKQRPEQGLEWIGWIDPENGKTICDPKFQGKASITADTSSNTAYLHLSSLTSEDTAVYYCATTWGVPYWGQGTLVTVSA

SEQ ID NO: 13 - protein, 8 aa, synthetic construct
  REGION 1..8: note = Synthetic: CDR1
GFNVKDYY

SEQ ID NO: 14 - protein, 9 aa, synthetic construct
  REGION 1..9: note = Synthetic: CDR2
IDPENGKTI

SEQ ID NO: 15 - protein, 8 aa, synthetic construct
  REGION 1..8: note = Synthetic: CDR3
ATTWGVPY

SEQ ID NO: 16 - DNA, 337 bp, synthetic construct
  misc_feature 1..337: note = Synthetic: Light Chain
  source 1..337: mol_type = other DNA
gatgttgtgatgacccagactccactcactttgccggttaccattggacaaccagcctccatctcttgcaagtcaagtcagagcctcttagatagtgatgggaggacatatttgaattggttgtcacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacactttccattcacgttcggctcggggacaaagttggaaataaaac

SEQ ID NO: 17 - protein, 112 aa, synthetic construct
  REGION 1..112: note = Synthetic: Light Chain
DVVMTQTPLTLPVTIGQPASISCKSSQSLLDSDGRTYLNWLSQRPGQSPKRLIYLVSKLDSGVPDRFTGSGSGTDFTLKISRVEAEDLGVYYCWQGTHFPFTFGSGTKLEIK

SEQ ID NO: 18 - protein, 11 aa, synthetic construct
  REGION 1..11: note = Synthetic: CDR1
QSLLDSDGRTY

SEQ ID NO: 19 - skipped

SEQ ID NO: 20 - protein, 9 aa, synthetic construct
  REGION 1..9: note = Synthetic: CDR3
WQGTHFPFT

SEQ ID NO: 21 - DNA, 358 bp, synthetic construct
  misc_feature 1..358: note = Synthetic: alpha-OmpW2-2 Heavy Chain
  source 1..358: mol_type = other DNA
gaggtgcagctgcaggagtctggaggaggcttggtacaacctggaggatccatgaaactctcctgtattgccyctggattcactttcagtaacgactggatgaactgggtccgccagtctccagagaaggggcttgagtgggttgctgaaattagattgaaatctaataattatgcaacacgttatgtggagtctgtgaaagggaggttcaccatctcaagagatgattccaaaagttctgtctacctgcaaatgaacaacttaagagctgaagacactggcatttattactgtaccaggccgggtaactggtacttcgatgactggggcgcagggaccacggtcaccgtctcctcag

SEQ ID NO: 22 - protein, 119 aa, synthetic construct
  REGION 1..119: note = Synthetic: alpha-OmpW2-2 Heavy Chain
EVQLQESGGGLVQPGGSMKLSCIASGFTFSNDWMNWVRQSPEKGLEWVAEIRLKSNNYATRYVESVKGRFTISRDDSKSSVYLQMNNLRAEDTGIYYCTRPGNWYFDDWGAGTTVTVSS

SEQ ID NO: 23 - protein, 8 aa, synthetic construct
  REGION 1..8: note = Synthetic: CDR1
GFTFSNDW

SEQ ID NO: 24 - protein, 10 aa, synthetic construct
  REGION 1..10: note = Synthetic: CDR2
IRLKSNNYAT

SEQ ID NO: 25 - protein, 10 aa, synthetic construct
  REGION 1..10: note = Synthetic: CDR3
TRPGNWYFDD

SEQ ID NO: 26 - DNA, 322 bp, synthetic construct
  misc_feature 1..322: note = Synthetic: Light Chain
  source 1..322: mol_type = other DNA
gacatcaaggtgacccagtctccatcttccatgtatgcatctcttggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatttgggaatttattattgtctacagtatgatgagtttccgctcacgttcggtgctgggaccaagctggagctgaaac

SEQ ID NO: 27 - protein, 107 aa, synthetic construct
  REGION 1..107: note = Synthetic: Light Chain
DIKVTQSPSSMYASLGERVTITCKASQDINSYLSWFQQKPGKSPKTLIYRANRLVDGVPSRFSGSGSGQDYSLTISSLEYEDLGIYYCLQYDEFPLTFGAGTKLELK

SEQ ID NO: 28 - protein, 6 aa, synthetic construct
  REGION 1..6: note = Synthetic: CDR1
QDINSY

SEQ ID NO: 29 - skipped

SEQ ID NO: 30 - protein, 9 aa, synthetic construct
  REGION 1..9: note = Synthetic: CDR3
LQYDEFPLT

SEQ ID NO: 31 - protein, 6 aa, synthetic construct
  REGION 1..6: note = Synthetic: BauA epitope
ADEPFA

SEQ ID NO: 32 - protein, 6 aa, synthetic construct
  REGION 1..6: note = Synthetic: OmpW2 epitope
QNVLDG

(19) United States
(12) Patent Application Publication
(10) Pub. No.: US 2026/0201022 A1
(43) Pub. Date: Jul. 16, 2026

(54) MONOCLONAL ANTIBODIES THAT INTERFERE WITH IRON UPTAKE

(71) Applicants: Daniel Vincent ZURAWSKI, Monrovia, MD (US); Mariel Giselle ESCATTE, Gaithersburg, MD (US); Yoann Stephane LE BRETON, Burtonsville, MD (US); THE UNITED STATES GOVERNMENT, AS REPRESENTED BY THE SECRETARY OF THE ARMY, Fort Detrick, MD (US)

(72) Inventors: Daniel Vincent ZURAWSKI, Monrovia, MD (US); Mariel Giselle ESCATTE, Gaithersburg, MD (US); Yoann Stephane LE BRETON, Burtonsville, MD (US)

(21) Appl. No.: 18/881,793

(22) PCT Filed: Jul. 24, 2023
(86) PCT No.: PCT/US2023/028447

§ 371 Date: Jan. 7, 2025

Related U.S. Application Data
(60) Provisional application No. 63/369,103, filed on Jul. 22, 2022.

(51) Int. Cl. C07K 16/1218; A61K 39/40; A61K 45/06; A61P 31/04

(52) U.S. Cl. CPC C07K 16/1218; A61K 39/40; A61K 45/06; A61P 31/04

(57) ABSTRACT

Two surface proteins of A. baumannii, which are required for iron transport of the bacteria, were identified: BauA, which is a siderophore receptor, and OmpW2, which are porins. Amino acid- and DNA sequences of monoclonal antibodies that bind to BauA and OmpW2 antigens were identified. These monoclonal antibodies were able to reduce bacterial growth and biofilm formation, and when they were applied together, they work in synergy. It was also demonstrated that these antibodies prevented infection in three separate animal models of infection. Thus, the invention provides pharmaceutically applicable compositions and methods of use thereof as well as a kit for outdoor use, based on these mouse monoclonal antibodies in order to develop human monoclonal antibodies against BauA and/or OmpW2 of A. baumannii as surface targets. These antibodies block their function and render the bacteria avirulent and bacteriostatic so it can be killed by the immune system.

### GOVERNMENT FUNDING SUPPORT

This invention was made with government support under grant no. W0350_20_WR awarded by the Military Infectious Diseases Research Program and previous awards from the same program. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Sep. 11, 2023, is named “15969-022PC0.xml” and is 40,549 bytes in size. The sequence listing contained in this .XML file is part of the specification and is hereby incorporated by reference herein in its entirety.

### BACKGROUND

### 1. Field of the Invention

The invention relates to the general field of antibodies and its pharmaceutical compositions, and in particular to monoclonal antibodies targeting bacterial surface proteins of Acinetobacter baumannii or antigen-binding fragment thereof for prophylactic and therapeutic treatment of A. baumannii infection.

### 2. Background of the Invention

Multidrug-resistant bacteria pathogens, the ESKAPEE (Enterococcus faecium, Staphylococcus aureus, Klebsiella pneumoniae, Acinetobacter baumannii, Pseudomonas aeruginosa, Enterobacter spp. and Escherichia coli), are responsible for over 10,000,000 infections a year worldwide and roughly 2.5 million deaths because of their high virulence and antibiotic resistance. Among them, Acinetobacter baumannii infections make up approximately 1,000,000 infections worldwide and are responsible for >30,000 excess deaths/year with high mortality rates (>50%) when strains are multidrug-resistant and occur in the intensive care unit (ICU)(Cavallo I, Oliva A, Pages R, Sivori F, Truglio M, Fabrizio G, Pasqua M, Pimpinelli F, Di Domenico EG. Acinetobacter baumannii in the critically ill: complex infections get complicated. Front Microbiol. 2023 Jun. 22; 14:1196774). In the US, it is estimated there are up to ~85,000 infections year with similar mortality rates associated with patients in the ICU (Wong D, Nielsen T B, Bonomo R A, Pantapalangkoor P, Luna B, Spellberg B. Clinical and Pathophysiological Overview of Acinetobacter Infections: a Century of Challenges. Clin Microbiol Rev. 2017 January; 30(1):409-447.)

Acinetobacter baumannii is one of the most common Acinetobacter species in clinical settings, which is a genus of Gram-negative bacteria including non-lactose-fermenting, catalase-positive, non-motile, non-fastidious, oxidase-negative, aerobic, and short, almost round, rod-shaped (coccobacillus) bacteria. Due to its remarkable ability to acquire antibiotic resistance, conferring multi-drug resistance (MDR), A. baumannii is one of the most challenging bacterial pathogens. For example, A. baumannii is able to survive regardless of the availability of dissolved oxygen once entering the environment, which represents a serious public health concern. (Raut S, Rijal K R, Khatiwada S, Kama S, Khanal R, Adhikari J, Adhikari B. Trend and Characteristics of Acinetobacter baumannii Infections in Patients Attending Universal College of Medical Sciences, Bhairahawa, Western Nepal: A Longitudinal Study of 2018. Infect Drug Resist. 2020 Jun. 8; 13:1631-1641.) In fact, there are some A. baumannii isolates that are resistant to all classes of antibiotics.

The clinical impact of A. baumannii infections has grown over the last twenty years. A. baumannii can cause infections in the blood, urinary tract, lungs, or in wounds in other parts of the body, leading to pneumonia, bacteremia, sepsis, amputations, and death, which is often due to treatment failure against multidrug-resistant and sometimes even pandrug-resistant strains ( )(Cavallo I, Oliva A, Pages R, Sivori F, Truglio M, Fabrizio G, Pasqua M, Pimpinelli F, Di Domenico EG. Acinetobacter baumannii in the critically ill: complex infections get complicated. Front Microbiol. 2023 Jun. 22; 14:1196774)). A. baumannii can sometimes be aggressive and detrimental with patients who have hospital-acquired pneumonia or ventilator-acquired pneumonia with indications of >50-800% mortality rates, and those patients can die within 2-3 days without the appropriate antibiotic treatment.

Since many strains are often multidrug- or pandrug-resistant now, doctors have very limited choices for treatment. Recently, the WHO identified A. baumannii as an urgent threat to public health. The lack of new antibiotics currently being pursued against A. baumannii as well as other gram-negative bacteria is of grave concern, given the increasing levels of antibiotic resistance. Therefore, alternative antibacterial approaches should be considered such as antibodies targeting the surface-expressed proteins required for virulence and pathogenesis.

Such an increase of multidrug- or pandrug resistant bacteria, including A. baumannii and other ESKAPEE pathogens, presents a serious problem, especially to wounded soldiers in battlefield. Unfortunately, the numbers of casualties are correlated with evacuation times because of bacterial infections and sepsis associated with the wound. As was learned from recent conflicts such as Operation Enduring Freedom (OEF), providing an antibiotic during the first hour of injury (termed the “Golden Hour”) can reduce the likelihood of developing a bacterial infection and result in better patient outcomes. Without rapid evacuation or early intervention, the rate of sepsis will likely be similar to what was observed in the Vietnam War. Sepsis was the third-leading cause of death (12%) in the first 24 hours, and after 24 hours, sepsis was the leading cause of death (38%) in wounded personnel. This coincides with delayed evacuations that were on average 3-7 days, which could be similar to what is projected with modern warfare.

It is also envisioned that most first-line treatments remediate these bacterial species. For example, prophylactic antibiotics provided in the current Individual First Aid Kit (iFAK) or medic's bag will prevent WWI-like conditions where gangrene (Clostridium) and anaerobic bacteria were a problem. The Current Practice Guidelines (CPG, ID:62) call for broad-spectrum, empiric antibiotic therapy such as cefazolin, moxifloxicin, or ertepenem to be administered by medics or by other caregivers throughout evacuation. Additionally, other broad-spectrum approaches found in the medic's bag such as silver-impregnated dressings could also be used by medics; however, these dressings do not treat the bacteria deep in wound bed or show efficacy against biofilms. Bacteria have also become resistant to silver, especially in polymicrobial settings. Therefore, as we have seen with recent conflicts, the ever-growing problem of antimicrobial resistance (AMR) will likely be a problem on the battlefield as well.

Recently, anti-bacterial bacteriophage approach seems to be promising as a salvage therapy against MDR-bacterial infection, however bacteriophages are strain-specific, and seem to only work with antibiotics. In addition, bacteriophages are cleared by the immune system, and therefore phages (and some small molecules with short half-lives) require repeated dosing over a 24 hr period, which would make it difficult for medics in a PFC situation to administer. Also, bacteriophage and antimicrobial peptides (another antibacterial approach) have never been approved by the FDA.

However, as it was recently shown with COVID-19 infection cases, one of the best methods to prevent infection or limit symptoms is using vaccines or monoclonal antibodies (mAbs) before or during infection. However, vaccination is not an ideal approach for wound infections and military application since vaccinating the whole US military is not cost-effective when only a small proportion are wounded. Thus, it may be better to use monoclonal antibodies (mAbs) in prophylactic and treatment settings.

### SUMMARY OF THE INVENTION

The use of antibodies to overcome MDR-bacterial infection has advantages over other antibacterial approaches. For example, with a bacteriophage approach, repeated dosing and the presence of antibiotics is necessary to remediate infection. In contrast, a similar efficacy can be seen with antibodies administered just by a one-time injection that can last weeks-months. In addition, the use of antibodies presents other several advantageous points. First, antibodies will not interfere with antibiotics or other medications, and therefore can be a tool for both the prevention and for the treatment of severe infections, which enhance patient outcomes. Second, antibodies are bacterial species specific and won't disrupt the protective microbiome. Third, they work with multiple mechanisms of action. Some mAbs can neutralize toxins secreted by the pathogens, others can inhibit growth, but most importantly, they can work with a patient's immune system to provide a means for opsonophagocytosis and complement activity. Fourth, antibodies can be produced relatively easily using recombinant technology and the use of mammalian cell lines, as was seen during the pandemic. In contrast, small molecules can require extensive, multi-step chemistry or expensive precursor molecules. Fifth, clinical safety is less of a concern. Small molecule antibiotics, in contrast, require extensive absorption, distribution, metabolism, excretion and toxicity (ADMET) studies, which increase drug development time and costs. Lastly, unlike vaccines that require a person's immune system to function properly, antibodies can just be delivered at a desired dosage, even in immunocompromised individuals to obtain a protective effect.

Therefore, this invention is useful in the field for: (1) Preventative measures for infectious diseases, to include clinical indications where A. baumannii is known organism responsible for said infections. These clinical indications and A. baumannii infections include but are not limited to: hospital-acquired and ventilator-associated pneumonia, urinary tract infections, skin and soft tissue infections, and necrotizing faciitis. Additionally, the invention could also be useful for preventative measures in combat wound care in austere and prolonged field care environments, and (2) Therapeutic measures for infectious diseases and clinical indications aforementioned, including combat wound care in austere and prolonged field care environments.

To this end, as an example for MDR-bacterial infection treatment, in this disclosure, two surface proteins expressed on the outer membrane of Acinetobacter baumannii, which are required for infection, were identified: BauA, which is a siderophore receptor on the surface of A. baumannii that sequesters iron from the environment for survival; and OmpW2, which is a porin, channel proteins on the outer membrane allowing transport of iron and nutrient acquisition in the host. Using hybridoma cells, amino acid sequences and DNA sequences of mouse monoclonal antibodies, αBauA and αOmpW2, that bind to these antigens were identified. These two antibodies, when used together, work in synergy to disrupt bacterial growth and biofilm formation and prevent infection in three separate animal models of infection. Further, it was demonstrated that αBauA and αOmpW2 antibodies recognized the native surface of the majority of strains of A. baumannii. Additionally, αOmpW2 recognizes OmpW1; therefore, this is a three-target approach with broad strain coverage.

Therefore, the present disclosure provides monoclonal antibodies against BauA and OmpW2 of Acinetobacter baumannii or antigen-binding fragments thereof for prophylactic or therapeutic treatment of A. baumannii infection. Further, the present disclosure provides a pharmaceutical composition comprising the monoclonal antibodies against BauA and/or OmpW2 or antigen-binding fragments thereof, method of using the pharmaceutical composition, and a kit comprising the pharmaceutical composition for field use or off-the-shelf use. Additionally, the antibodies of this disclosure can be used singly or in combination or with other antibodies or antibiotics to treat infections. These antibodies can also be used in conjunction with antibiotics to treat an already established infection.

For this purpose, in this disclosure were identified sequences of heavy chain variable regions and light chain variable regions of monoclonal anti-BauA and anti-OmpW2 antibodies, as well as CDR sequences, which can be grafted to human antibody to generate humanized antibody.

For this invention, it can also be contemplated to use these mouse monoclonal antibodies for the development of human monoclonal antibodies against these targets to avoid Human Anti-Mouse Antibodies (HAMA) responses.

Another advantage is that the idea of this invention can be applied to other ESKAPEE pathogens. Instead of making new antibiotics, using monoclonal antibodies to prevent infections is a novel approach. This approach could limit mortality and morbidity in the hospital environment if given before surgery or entry to ICU.

### BRIEF DESCRIPTION OF THE DRAWINGS

The following drawings form part of the present specification and are included to further demonstrate certain embodiments of the present disclosure. The invention may be better understood by reference to one or more of these drawings in combination with the detailed description of specific embodiments presented herein.

FIG. 1. Original depiction of the idea (ca. 2009) generating antibodies that attack iron acquisition and mixing these antibodies with treatments such as iron chelators as a treatment for bacterial infection (open fracture wound infection model).

FIG. 2. Flow chart for obtaining monoclonal antibodies.

FIG. 3. OmpW1 polyclonal antibody (ca.2010) recognized the surface of AB5711, a strain of A. baumannii (left).

FIG. 4. A. baumannii AB5075 (A. baumannii model strain) were subcultured in log phase into lysogeny broth (LB) in a 96-well plate. Wells were treated with 0.25 the minimal inhibitory concentration of an iron chelator (VK28) along with 5.0 μg/mL of OmpW1 Abs (orange), OmpW2 (gray) Abs or the combination (yellow) of both. Combination of OmpW1 and OmpW2 polyclonal antibodies appeared to have a synergistic effect.

FIG. 5. A bauA transposon mutant (Gallagher et al. 2015) was attenuated for virulence in a murine pulmonary model of infection when compared to wild-type mice. N=10

FIG. 6. In iron-deficient medium (ID-CAMHB), both αBauA and αOmpW2 monoclonal antibodies reduced growth of A. baumannii AB5075, αBauA (red) and αOmpW2 (blue), and the combination of both antibodies and synergy (purple) is much more efficacious. In nutrient-rich medium, this effect was not apparent (left).

FIG. 7. A. baumannii were grown in 96-wells with pegs where biofilms were formed over 24-48 hours. αBauA (red) or αOmpW2 (orange) was provided at 100 μg/mL each or together (purple) at 50 μg/mL each, and their effect on biofilm formation by A. baumannii was compared with that by A. baumannii untreated (light blue) or isotype antibody (αHIV-envelope) controls. Biofilms were measured by OD₅₈₀/OD₆₀₀ ratio which takes into account mass/growth.

FIG. 8. Native ELISA was performed where A. baumannii strain AB5075 were grown to log phase, seeded, and dried on ELISA plates. Each antibody: αHcp (green), αBauA (red) and αOmpW2 (blue) passed over each well and incubated with bacteria. After an hour, secondary antibody conjugated to HRP was incubated with each well. Developer was added and wells were measured in a plate reader at OD₄₅₀.

FIG. 9. αBauA (top) and αOmpW2 (bottom) recognized 68% and 88% of total strains of A. baumannii from the diversity set, suggesting the targets are well-conserved.

FIG. 10. G. mellonella waxworms. (A) Live (light) and dead larvae (dark) can be differentiated by melanization. In contrast to the control group (far left), groups treated with monoclonal antibodies prevented death (right). (B). Kaplan-Meier survival curve over 48 hours evaluating G. mellonella infected with A. baumannii AB5075. Single mAb applications provide some protection, but the combination provides 90% and 80% protection depending on the dose, 2.5 μg/mL and 5.0 μg/mL respectively.

FIG. 11. Kaplan-Meier curves of representative murine pulmonary model experiment (n=10) repeated three times. The combination of αBauA and αOmpW2 provided 70% protection on Day 4/5 when compared with single-treated or untreated mice and 50% protection over 8 days. Data also suggests efficacy is dose-dependent.

FIG. 12 (A) Wound closure measurement over 23 days. When infected wound was treated with both antibodies (40 mg/kg) prophylactically on the day before infection, wound size is dramatically reduced (>70%) on Day 23. Relative size in wound bed suggests reduced inflammation after antibody treatment. (B) Kaplan-Meier curves comparing the control group vs. the treatment group in the murine model of wound infection. When some animals succumbed to infection (40% survival for the isotype (αOvalbumin antibody)-treated animals), only the animals that received the antibodies survived 100%.

FIG. 13. Western blot analyses with αOmpW2 monoclonal antibody. (A) Purified H6-OmpW2 was run by SDS-PAGE, transferred onto nitrocellulose and hybridized with the αOmpW2 antibody, revealing the target peptide sequence is likely linear. (B) Additional Western blot analyses were carried out using cell lysates of E. coli strains expressing recombinant GST-OmpW1 and GST-OmpW3.

FIG. 14 (A) Proposed 3D structures of BauA. Shaded region is the surface exposed region of protein, 100 amino acids where the epitope for αBauA resides. (B) Proposed 3D structures of OmpW2. Shaded region is the surface exposed region of protein, 100 amino acids where the epitope for αOmpW2.

FIG. 15—Overlapping peptides determined the epitope for αBauA using Gator Plus instrument. Highlighted in red with predicted structure. Epitope and structure would be exposed on the surface and is consistent with the antibody disrupting function.

FIG. 16—Overlapping peptides determined the epitope for αOmpW2. Highlighted in red with predicted structure. Epitope and structure would be exposed on the surface and is consistent with the antibody disrupting function.

FIG. 17. Antibody sequences of αOmpW2-2 and αBauA, and IgG illustration.

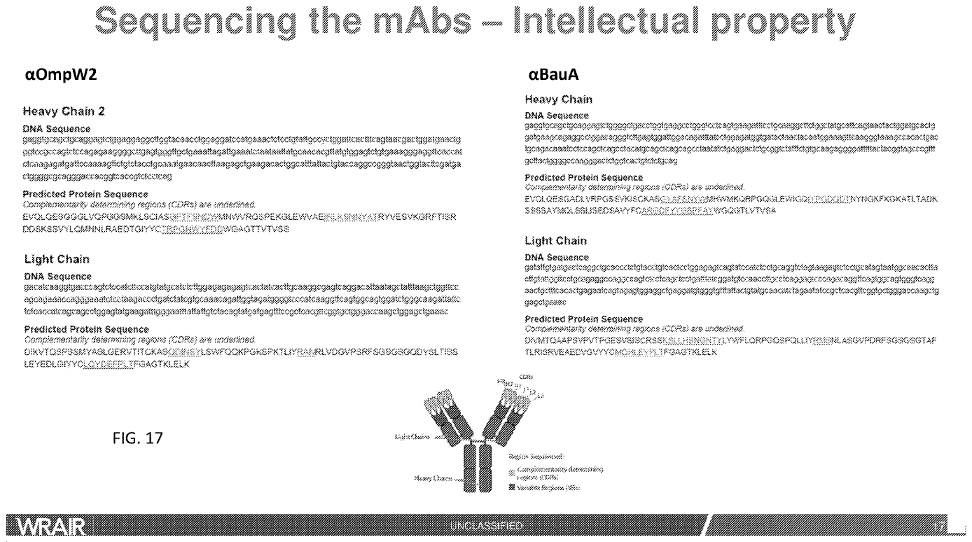

### DETAILED DESCRIPTION

In the Summary above, in the Detailed Description, and the claims below, as well as the accompanying figures, reference is made to particular features of the invention. It is to be understood that the disclosure of the invention in this specification includes all possible combinations of such particular features. For example, where a particular feature is disclosed in the context of a particular embodiment or embodiment of the invention, or a particular claim, that feature can also be used, to the extent possible, in combination with and/or in the context of other particular embodiments and embodiments of the invention, and in the invention generally. For the purposes of explanation, numerous specific details are set forth in order to provide a thorough understanding of the present disclosure. It will be apparent, however, to one skilled in the art that the present invention may be practiced without these specific details.

### Definitions

Embodiments of materials and methods are described herein; any methods and materials similar or equivalent to those described herein can be used in the practice of or testing of the invention. Unless defined otherwise, all technical and scientific terms herein have the same meaning as commonly understood by one of ordinary skill in the art to which the invention pertains. In describing and claiming the present invention, the following terminology will be used. It is also to be understood that the terminology used herein is for the purpose of describing particular embodiments only, and it is not intended to be limiting.

As used herein and in the appended claims, the singular forms “a,” “an,” and “the” include plural references unless specifically stated otherwise.

Other than in the operating examples, or where otherwise indicated, all numbers expressing quantities of ingredients or reaction conditions used herein should be understood as modified in all instances by the term “about.” The term “about” generally refers to a range of numbers that one of skill in the art would consider equivalent to the recited value (i.e., having the same function or result). In a specific embodiment, the term “about” includes a stated numerical value as well as a value that is +/−15% of the stated numerical value. For example, about 5.75 M includes 5.75 molar as well as 6.61 M and 4.89 M, and all 1/10 values in between. In many instances, the term “about” may include numbers that are rounded to the nearest significant figure.

As used herein, the term “immune system” refers to the cells and molecules responsible for immune responses, and the term “immune response” refers to their collective and coordinated reactions to exposure to foreign substances introduced into the body. The immune system is made up of two parts: innate immune system (also called natural immunity or native immunity) and adaptive immune system (also called specific immunity or acquired immunity). Innate immune system reacts almost immediately to the exposure to foreign microbes and damaged cells, and repeated exposures induce very similar innate immune responses each time. The principal components of innate immune system include (1) physical and chemical barriers (e.g., epithelia), (2) phagocytic cells (e.g., neutrophils, macrophages), dendritic cells, mast cells, natural killer cells, and other innate lymphoid cells, and (3) blood proteins (e.g., components of the complement system and other mediators of inflammation). The adaptive immune system recognizes and reacts to various antigens of microbial and nonmicrobial substances, and mediated by lymphocytes, especially B lymphocytes and T lymphocytes. There are two types of adaptive immunity: cell-mediated immunity and humoral immunity. Cell mediated immunity (also called cellular immunity) is mediated by T lymphocytes. Humoral immunity is mediated by B cells, which recognize antigens and proliferate, differentiate, and secrete molecules into the blood and mucosal secretions, called antibodies., (Cellular and Molecular Immunology, 10th Edition, Abul K Abbas, Andrew H. Lichtman, and Shiv, Pillai, 2022 Elsevier)

As used herein, the term “active immunity” refers to the form of immunity induced by exposure to antigens, and the immune system of an antigen-exposed individual actively induce immune responses to the antigen.

As used herein, the term “passive immunity” refers to the form of immunity conferred on an individual, who has not been exposed to an antigen, by transferring antibodies from an immunized individual. The recipient of such antibodies become immune to the particular antigen without ever encountering or having responded to that antigen.

As used herein, the term “antigen” refers to the foreign molecules that stimulate immune responses.

As used herein, the term “epitope” or “antigenic determinant” refers to the part of complex antigen that is specifically recognized by antibodies and/or lymphocytes. Epitopes are generally divided into two categories, linear epitopes where a stretch of continuous amino acid residues are sufficient for recognition and binding, and conformational epitopes in which key amino acid residues that are discontinuous in the unfolded protein are brought together by protein folding to form an antigenic surface on the protein's three-dimensional structure.

As used herein, the term “antibodies” (also called immunoglobulin, Ig) refers to circulating globular proteins produced by B cells in response to exposure to antigens, and are the mediators of humoral immunity against all classes of antigens, including microbes. Antibodies recognize and bind to antigen molecular structures, more specifically, epitopes, neutralize and target the antigen for elimination by phagocytosis and the complement system.

Generally, antibody molecules share the same basic structure of the heterotetrameric glycoprotein. An antibody molecule has a symmetric core structure composed of two identical light chains and two identical heavy chains. Each light chain is linked to a heavy chain by one covalent disulfide bond between the carboxy terminus of the light chain and the C_{H}1 domain of the heavy chain, while the two heavy chains are linked to each other by one or more disulfide bonds, depending on the heavy chain isotype. Each heavy and light chain also has regularly spaced intrachain disulfide bridges. Each heavy chain and light chain both consists of an amino-terminal variable (V) region that participates in antigen recognition and a carboxy-terminal constant (C) region; the C regions of the heavy chains are glycosylated, interact with other molecules and cells of the immune system and help mediate most of the effector functions of antibodies.

Depending on the differences in the structure and amino acid sequence of the C region domain of the heavy chains (C_{H}), antibodies can be divided into five classes of immunoglobulin isotypes: IgA, IgD, IgE, IgG, and IgM, having heavy chains designated α, δ, ε, γ, and μ, respectively. The α, γ and δ isotype heavy chains have three constant domains (C_{H}) (from amino terminus to carboxy terminus, C_{H}1, C_{H}2, C_{H}3), and μ and ε isotype heavy chains have four C_{H} domains.

In humans, IgA and IgG isotypes can be further subdivided into closely related subclasses, or subtypes, called IgA1 and IgA2, and IgG1, IgG2, IgG3, and IgG4. (Mice differ from humans in that the IgG isotype is divided into the IgG1, IgG2a, IgG2b, and IgG3 subclasses; certain strains of mice, including C57BL/6, lack the gene for IgG2a but produce a related isotype called IgG2c.) Based on the amino acid sequences of the C region domain of the light chains (C_{L}), the light chains can be divided into two clearly classes, or isotypes, called κ and λ.

In addition, all isotypes of heavy chains can be expressed either as secreted immunoglobulin or immunoglobulin anchored to the plasma membranes of B lymphocytes, which differ at their carboxy-terminal ends. The C regions of light chains do not participate in effector functions and are not directly attached to cell membranes.

In the heavy chains, the V region is composed of one Ig domain (about 110 amino acids), and the C region is composed of three or four Ig domains. Each light chain is composed of one V region Ig domain and one C region Ig domain.

The V region of one heavy chain (V_{H}) and the V region of one light chain (V_{L}) form an antigen binding site, and IgG, IgE, and IgD antibodies usually have two antigen binding sites. (Secreted IgM antibodies consist of 5 of the basic heterotetramer unit along with an additional polypeptide called J chain, and therefore contain 10 antigen binding sites, while secreted IgA antibodies can polymerize to form polyvalent assemblages comprising 2-3 of the basic 4-heterotetramer unit along with J chain). In the case of IgGs, the heterotetramer unit is generally about 150,000 daltons. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, CT, 1994, page 71 and Chapter 6.

In the V regions of heavy chains and light chains, the variability is not evenly distributed across the 110-amino acid span. Instead, the V regions comprises 4 relatively invariant stretches called “framework regions” (FRs) of 15-30 amino acids, largely adopting a 3-sheet configuration, separated by 3 regions of extreme variability called “hypervariable regions” of 9-12 amino acids, which form loops connecting, and in some cases forming part of, the 3-sheet structure. Because these sequences are brought together to form an antigen binding surface of the antibody, the hypervariable regions are also called complementarity-determining regions (CDRs). Therefore, proceeding from either the V_{L} or the V_{H} amino terminus, these regions comprise the domains FR1, CDR1, FR2, CDR2, FR3, CDR3, and FR4.

As used herein “polyclonal antibody” refers to a collection of antibodies that are secreted by multiple different B cell lineages and recognize different epitopes on the same antigen.

As used herein “monoclonal antibody” refers to a collection of antibodies that are produced by the progeny of a single B cell clone which are clones of a single parent cell, and therefore all the antibody molecules secreted by those cells have the same V region and bind to the same epitope of the antigen that originally triggered the B cells. This means that the antibody recognizes only a single epitope of an antigen and is extremely specific. The monoclonal antibodies in this disclosure may be prepared by immunizing the mouse or using the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J, Mol, Biol., 222:581-597 (1991), for example.

As used herein, the term “hybridoma” refers to a culture of hybrid cells that results from the fusion of B cells and myeloma cells, which is a tumor of plasma cells, for the generation of monoclonal antibodies. In this procedure, first a mouse is immunized with a known antigen or mixture of antigens, and then spleen B cells are isolated and fused with an enzyme-deficient partner myeloma cells in the presence of chemicals such as polyethylene glycol, which can facilitate the fusion of the plasm membranes of two different cell types (B cells and myeloma cells here) to form hybridoma cells that obtain many chromosomes from both fusion partner cells. The myeloma partner chosen for hybridoma cell generation is the cell line that does not secrete its own immunoglobulin (Ig). These fusion cells of B cells and myeloma cells (hybrid cells) are cultured in a selection medium, which allows the survival of only immortalized hybrids. Then, these hybrid cells are separated individually, cultured as single cell clones, and tested for the secretion of the antibody targeting a specific epitope. The selection medium has hypoxanthine, aminopterin, and thymidine (HAT medium). Most cells utilize two pathways for purine synthesis, a de novo pathway that requires tetrahydrofolate to deliver formyl group, and a salvage pathway that uses the enzyme hypoxanthine-guanine phosphoribosyl-transferase (HGPRT). As a hybrid fusion partner, myeloma cells that do not have HGPRT are used, which normally survive using de novo purine synthesis. In the presence of aminopterin, dihydrofolate reductase (DHFR) is inhibited, which catalyzes the reduction of DHF to THF in the presence of NADPH, and consequently tetrahydrofolate is not made, resulting in a defect in de novo purine synthesis as well as a specific defect in pyrimidine synthesis, for example, the synthesis of thymidine monophosphate (TMP) from deoxyuridine monophosphate (dUMP). Hybrid cells that have received HGPRT from the spleen B cells also acquire the capability of uncontrolled proliferation from the myeloma partner; when these hybrid cells are cultured in the presence of hypoxanthine and thymidine, these cells can make DNA in the absence of tetrahydrofolate. As a result, only hybrid cells can survive in HAT medium. Each hybridoma makes only one type of Ig, derived from one B cell from the immunized animal. (Cellular and Molecular Immunology, 10th Edition, Abul K Abbas, Andrew H. Lichtman, and Shiv, Pillai, 2022 Elsevier)

Monoclonal antibodies produced by this way may be purified/isolated, if desired, using filtration, centrifugation and various chromatographic methods such as Protein G or A affinity chromatography or FPLC, using assay of binding and neutralization. Generally, complete antibodies are fractionated utilizing agents (i.e., protein A or protein G) that bind the Fc portion of the antibody. Alternatively, antigens fixed to a support, such as beads or resins packed in a column may be used to simultaneously purify and select appropriate antibodies. After antibody binding to the protein A/G or antigen fixed to a support, contaminants are removed (e.g., washed away), and the antibodies are released by applying conditions (salt, heat, etc.).

The fragments of monoclonal antibody of the present disclosure can be obtained from the purified/isolated monoclonal antibodies by methods that include digestion with enzymes, such as pepsin or papain, and/or by cleavage of disulfide bonds by chemical reduction. Alternatively, the fragments of monoclonal antibody encompassed by the present disclosure can be synthesized using an automated peptide synthesizer.

It also is contemplated that a molecular cloning approach may be used to generate monoclonal antibodies. For this, hybridomas may be cultured, then cells lysed, and total RNA extracted. Random hexamers may be used with reverse transcriptase to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Alternatively, combinatorial immunoglobulin phagemid libraries are prepared from RNA isolated from the cell lines and phagemids expressing appropriate antibodies are selected by panning using viral antigens.

Monoclonal antibodies according to the present disclosure may be defined, in the first instance, by their binding specificity which in this case is for BauA and OmpW2 proteins. Those of skill in the art, by assessing the binding specificity/affinity of a given antibody using techniques well known to those of skill in the art, can determine whether such antibodies fall within the scope of the instant claims.

As used herein, the term “chimeric antibody” refers to a monoclonal antibody of structural chimera comprising variable regions from one species like a mouse, fused to constant regions from another species such as a human being, as well as fragments of such antibodies, so long as they exhibit the desired biological activity (see U.S. Pat. No. 4,816,567; and Morrison et al., Proc. Natl, Acad. Sei. USA, 81:6851-6855 (1984)). For example, chimeric antibodies can be “primatized” antibodies comprising amino acid sequences of variable regions derived from a non-human primate (e.g., monkey, ape etc.) and amino acid sequences of constant regions derived from human. Chimeric antibodies are usually prepared by recombinant DNA techniques. For example, chimeric antibodies comprising murine variable regions and human constant regions are the product of expressed immunoglobulin genes comprising DNA segments encoding murine immunoglobulin variable regions and DNA segments encoding human immunoglobulin constant regions. Other forms of “chimeric antibodies” encompassed by the present disclosure are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. See, e.g., Morrison, S. L., et al., Proc. Natl. Acad Sei. USA 81 (1984) 6851-6855; U.S. Pat. Nos. 5,202,238 and 5,204,244.

As used herein, the term “antibody fragment” refers to an antibody fragment or a synthetic polypeptide comprising an intact or partial antigen binding capacity of variable regions of an antibody. Examples of antibody fragments include Fab fragment, Fab′ fragment, F(ab′)₂ fragment, Fv fragment, disulfide-stabilized Fv fragment (dsFv), single-chain antibody (scFv or sFv) and (scFv)₂, diabody, nanobody, humabody, and multispecific antibody formed from antibody fragments (see U.S. Pat. No. 5,641,870, Example 2; Zapata et al., Protein Eng, 8(10): 1057-1062 [1995]).

As used herein, the term “Fab fragment” refers to a fragment comprising an antigen binding region of an antibody molecule. Papain digestion of an IgG antibody allows separation of two antigen binding regions (2 Fab fragments, each comprising V_{L} and C_{L}, and V_{H} and C_{H}1) of about 50 kDa each from the Fc region (C_{H}2 and C_{H}3) that is the carboxy terminal regions of two heavy chains held together by disulfide bonds and involved in effector functions of the antibody, such as binding to complement and Fc receptors (the Fc fragment) found on certain types of immune cells. The Fab fragment consists of the variable domain (V_{L}) and constant domain (C_{L}) of a light chain (an entire light chain), and the variable domain of a heavy chain (V_{H}) and the first constant domain of a heavy chain (C_{H}1). Each Fab fragment is monovalent with respect to antigen binding, i.e., it has a single antigen-binding site.

As used herein, the term “F(ab′)₂ fragment” refers to a fragment generated by pepsin digestion of a whole IgG antibody. Pepsin digestion removes most of the C_{H}2 and C_{H}3 regions except for a few residues at the carboxy terminuses of the C_{H}1 domains of the two heavy chains, which is called a hinge region having two disulfide bonds linking two intact antigen-binding F(ab) portions together. F(ab′)₂ fragment is a single bivalent antigen binding fragment with a molecular weight of about 110 kDa. Other chemical couplings of antibody fragments are also known.

As used herein, the term “Fab′ fragment” refers to a fragment generated by treating the F(ab′)₂ with β-mercaptoethanol, which reduces the cysteine disulfide bonds between the carboxy terminuses of the two heavy chain constant (C_{H}1) domains to generate a Fab′-SH fragment, which is monovalent.

As used herein, the term “Fv fragment” (25,000 daltons) refers to the smallest fragment generated from IgG and IgM that contains a complete antigen binding site. Fv fragments comprises a dimer of one heavy- and one light-chain variable region domain in tight, non-covalent association, and have the same binding properties and similar three-dimensional binding characteristics as Fab.

As used herein, the term “disulfide-stabilized Fv fragment” (dsFv) refers to a molecule in which the V_{H}-V_{L} heterodimer is stabilized by an interchain disulfide bond engineered between structurally conserved framework positions distant from complementarity-determining regions (CDRs).

As used herein, the term “single-chain variable fragment” (scFv or sFv) refers to a fusion protein comprising the variable regions of the heavy (V_{H}) and light (V_{L}) chains of an antibody, covalently linked by a single polypeptide chain of 10 to about 25 amino acids such that the V_{H} and V_{L} can associate in a “dimeric” structure analogous to that in a two-chain Fv fragment. The linker is usually rich in glycine for flexibility, as well as serine or threonine for solubility, and can either connect the N-terminus of the V_{H} with the C-terminus of the V_{L}, or vice versa (Schirrmann, Thomas (8 Nov. 2004). “Tumorspezifisches Targeting der humanen Naturlichen Killerzellinie Y T durch Gentransfer chimärer Immunglobulin-T-Zellrezeptoren” (in German). Berlin. doi:10.18452/15246). From the folding of these two domains emanate six hypervariable loops (3 loops each from the V_{H} and V_{L} chain) that contribute the amino acid residues for antigen binding surface of an antibody. This protein retains the specificity of the original immunoglobulin, despite removal of the constant regions and the introduction of the linker. However, even a single variable domain from V_{H} or V_{L} (comprising only three CDRs specific for an antigen) has the ability to recognize and bind antigen, although at a lower affinity than the entire binding site. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

As used herein, the term “diabody” refers to a noncovalent dimer of single-chain Fv (scFv) fragments, in which V_{H} and V_{L} domains form inter-chain pairing (cross-over pairing), and therefore it is bivalent or bispecific. A scFv fragment (in which V_{H} and V_{L} domains form intra-chain pairing to be monovalent, see preceding paragraph) is prepared with a short linker (about 5-10 residues) between the V_{H} and V_{L} domains such that inter-chain but not intra-chain pairing of the V_{H} and V_{L} domains is achieved. Diabodies can be monospecific and bivalent by forming homodimers, and simultaneously bind two identical antigen epitopes. On the other hand, diabodies can be heterodimers of two different scFvs to be bispecific and monovalent, having two different Fv domains originating from two different antibodies binding different antigen epitopes so as to bridge different antigens leading to their hetero-dimerization or different epitopes on the same antigen. Another form of diabody is (scFv)₂, in which two scFv fragments are covalently linked to each other. Diabodies are described more fully in, for example, EP 404,097; WO 93/11161; Hudson et al., Nat. Med. 9:129-134 (2003); and Hollinger et al., Proc. Natl. Acad. Sci. USA, 90:6444-6448 (1993).

As used herein, the term “humabody” refers to a V_{H} fragment, which is the smallest part of an antibody capable of specific antigen binding, about a tenth the size of a standard antibody. This small size enables superior tissue accumulation and penetration, which translates to better therapeutic index compared with conventional IgGs.

As used herein, the term “nanobody” refers to a recombinant V_{H}-only antibody, with many unique properties such as small size, excellent solubility, superior stability, quick clearance from blood, and deep tissue penetration.

As used herein, the term “humanized antibody” refers to a type of antibodies made in the laboratory by combining a human antibody with a small part of a mouse or rat monoclonal antibody. For example, a CDR of murine antibody variable regions is grafted into the framework region of a human antibody. (See, e.g., Riechmann, L. et al., Nature 332 (1988) 323-327; and Neuberger, M. S. et al., Nature 314 (1985) 268-270.) One of the limitations of monoclonal antibodies for therapy is that these antibodies are most easily produced by immunizing a mouse, but patients treated with mouse antibodies will make antibodies against the mouse Ig, called human anti-mouse antibody (HAMA), which causes severe allergic reactions to the mouse antibodies that can range from a mild form, like a rash, to a more extreme and life-threatening response, such as kidney failure. These anti-Ig antibodies block the function or enhance clearance of the injected monoclonal antibodies. Genetic engineering techniques have been used to replace the sequences in the mouse monoclonal antibody with human sequences, and thereby avoid an anti-Ig response. The complementary DNAs (cDNAs) that encode the polypeptide chains of monoclonal antibody can be isolated from hybridoma, and these genes can be manipulated in vitro. Only small portions of the antibody molecule are responsible for binding to antigen (i.e., CDR1, 2, and 3 regions); the remainder of the antibody molecule are relatively less variable framework regions 1, 2, 3, and 4 in the variable regions and constant regions. These structural organization allows the DNA segments encoding the antigen binding sites from a mouse monoclonal antibody (donor antibody) to be inserted into a cDNA encoding a human antibody (recipient antibody), creating a hybrid gene. When it is expressed, the resultant protein retains the antigen specificity of the original mouse monoclonal antibody but has the core structure of a human Ig.

The antigen binding region (or hypervariable region) to be grafted into human antibodies can be a mammal, for example, mouse, rat, rabbit, cow, pig, goat, sheep, donkey, horse, camelid, non-human primate, or human, having the desired antibody specificity, affinity, and capability. In some instances, framework region (FR) residues of the non-human immunoglobulin are replaced by corresponding human residues. Furthermore, humanized antibodies may comprise residues that are not found in the recipient antibody or in the donor antibody. These modifications are made to further refine antibody performance. In general, the humanized antibody may comprise one or preferably two variable domains, comprising all or substantially all of the CDR1, 2, and 3 hypervariable loops corresponding to those of a non-human antibody and all or substantially all of the FRs of a human antibody. For further details, see Jones et al., Nature 321:522-525 (1986); Riechmann et al., Nature 332:323-329 (1988); and Presta, Cuff, Op, Struct, Biol, 2:593-596 (1992). See also the following review articles and references cited therein: Vaswani and Hamilton, Ann. Allergy, Asthma and Immunol., 1:105-115 (1998); Harris, Biochem. Soc. Transactions, 23:1035-1038 (1995); Hurle and Gross, Curr. Op. Biotech., 5:428-433 (1994).

As used herein, the term “human antibody” refers to antibodies having variable and constant regions derived from human germline immunoglobulin sequences.

Chimeric antibodies contain human light chain and heavy chain Fc sequences, and are approximately 70% similar to human antibodies, which reduces immunogenicity against mouse antibodies, but does not completely eliminate HAMA reactions. Humanized antibodies possess 85-90% human antibody sequences (Fc sequences and framework sequences of the variable regions), but the generation of humanized antibodies is more technically challenging than the generation of mouse-human fusion chimeric antibodies.

Human antibodies can be produced using a display method, in which human antibodies or antibody fragments against a specific epitope are displayed on the surface of bacteriophage, bacteria, yeast or other simple organisms. Phage display is a molecular biology technique to express and display human proteins on the surface of phages by modifying phage genomes in such a way that the coat proteins of bacteriophage virions are fused to human proteins or peptides of interest. On the surface of each bacteriophage, one type of synthetically created antigen-binding site of human Ig is displayed. By screening millions of such synthetic antibodies displayed on the phages for binding to the target epitope or antigen of interest, an antibody specific for the target epitope or antigen can be identified. However, there are some disadvantages of the phage display system. First, the library contains fragments instead of full-length antibodies, i.e., the antibody sequences expressed on the surface of phages are “based” on human sequences instead of “being” actual human sequences. Second, the proteins are expressed by bacteria or yeast cells, and considering differences in the expression system and post-translational modification in the bacterial and yeast cells, they may not be optimal options for use in human patients.

Human antibodies can also be produced from transgenic mice that are genetically modified to produce human antibodies by introducing human immunoglobulin loci into the germline of mice. Oocytes microinjection technology and the embryonic stem (ES) cells can also be utilized to this end. This technology, however, has its limitation; only antibodies that the immune system of the mouse recognizes can be produced.

Human antibodies can also be produced from B cells isolated from humans; however, this method has also its limitations, i.e., screening large numbers of candidate individuals to identify circulating antibodies against an antigen of interest. In addition, as with transgenic animal models, only antibodies against target antigens recognized by human immune system can be produced.

Recently, an immunologix platform technology has been developed to overcome these disadvantages. Using the technology, it is possible to produces human IgG antibody libraries. In this technology, antibodies are produced from naïve B cells isolated from human tonsil tissue stimulated by virtually any antigens. Since the antibodies are produced by human cells from human genes, they are 100% human antibodies. However, since primary B cells do not proliferate readily ex vivo, the B cells need to be transformed to be maintained in a long-term cell culture. Through ex vivo antigen stimulation, the B cells class-switch and may undergo somatic hypermutation, thus producing a human “library” of different IgG antibodies that can then be screened against any antigen. Thereby, the Immunologix platform generates antibody repertoire of vast diversity comparable with phage or yeast display. Furthermore, this technology utilizes full-length human IgG rather than bacterial or yeast expressed Fv fragments. Since no mouse or human immunization screening nor chimeric or humanization steps is necessary because the antibodies are produced from human B cells ex-vivo from full-length antibody libraries, this platform can reduce both the cost and time involved in producing human monoclonal antibodies. (Duvall M, Bradley N, Fiorini R N. A novel platform to produce human monoclonal antibodies: The next generation of therapeutic human monoclonal antibodies discovery. MAbs. 2011 March-April; 3(2):203-8.)

As used herein, the term “antibiotics” refers to medicines that treat bacterial infections in people and animals. They work by killing the bacteria or inhibiting their growth. According to Merck Manual (https://www.merckmanuals.com/home/infections/antibiotics/overview-of-antibiotics), they can be classified into the classes of aminoglycosides, carbapenems, cephalosporins, fluoroquinolones, glycopeptides and lipoglycopeptides (such as vancomycin), macrolides (such as erythromycin and azithromycin), monobactams (aztreonam), oxazolidinones (such as linezolid and tedizolid), penicillins, polypeptides, rifamycins, sulfonamides, streptogramins (such as quinupristin and dalfopristin) and tetracyclines

As used herein, the term “multidrug resistance” refers to antibacterial resistance which happens when bacteria develop the ability to defeat the drugs designed to kill them. That means bacteria are not killed and continue to grow. CDC typically uses this term to refer to an isolate that is resistant to at least one antibiotic in three or more antibiotics classes. Multidrug resistance mechanisms fall into four main categories: (1) limiting uptake of a drug; (2) modifying a drug target; (3) inactivating a drug; (4) active drug efflux. There are two main ways that bacterial cells can acquire antibiotic resistance. One is through mutations that occur in the DNA of the cell during replication. The other way that bacteria acquire resistance is through horizontal gene transfer through plasmids or transposons coding for resistance to a specific agent. Examples of bacteria resistant to antibiotics are methicillin-resistant Staphylococcus aureus (MRSA), vancomycin-resistant Enterococcus (VRE), multi-drug-resistant Mycobacterium tuberculosis (MDR-TB) and carbapenem-resistant Enterobacteriaceae (CRE) gut bacteria.

As used herein, the term “subject” which can be used interchangeably with “patient” refers to an animal, preferably a mammal, or a human individual, which is to be treated with pharmaceutical compositions comprising antibodies or fragments thereof as taught herein. A “subject in need” for the invention can be an animal, preferably a human, that has been infected with A. baumannii or will be possibly infected with A. baumannii in the very near future.

As used herein, the terms “treat” refers to providing any type of medical management to a subject. Treating includes, but is not limited to, administering one or more drugs or agents comprising active ingredients to a subject using any known method for purposes such as alleviation or amelioration of one or more symptoms or conditions; diminishment of extent of disease, disorder, or condition; stabilization (i.e., not worsening) of a state of disease, disorder, or condition; prevention of spread of disease, disorder, or condition; delay or slowing the progress of the disease, disorder, or condition; amelioration or palliation of the disease, disorder, or condition; and remission (whether partial or total), whether detectable or undetectable.

As used herein, the term “pharmaceutically acceptable carrier” refers to “carrier excipient”. Pharmaceutical carrier or excipients are the substances other than the active ingredient(s), used in pharmaceutical dosage forms. They are considered as inert substances, i.e., they do not have any active role in therapeutics, but they can be used to support the process to produce an effective product.

As used herein, the term “excipients” refers to substances that are added to therapeutic products to improve stability, bioavailability, and manufacturability. Examples of excipients are preservatives to prevent contamination (e.g., thimerosal), adjuvants to help stimulate a stronger immune response (e.g., aluminum salts), stabilizers to keep the 3D structure of antibodies during transportation and storage (sugar, starch, gelatin, cellulose, cellulose derivatives, polyvinylpyrrolidone, and polyethylene glycol), surfactants to prevent antibody protein aggregation, amino acids, antioxidants, buffer (acetate, citrate, histidine, succinate, phosphate, and hydroxymethylaminomethane (Tris).), lyoprotectant (e.g., disaccharides such as sucrose and trehalose), and other additives including mannitol, BSA, serum, and skim milk.

As used herein, the term “dosage” refers to the administering of a specific amount, number, and frequency of doses over a specified period of time, and the term “dose” refers to a specified amount of medication taken at one time. A “dosage regimen” refers to the number of doses of a drug, medication, or an agent that a patient is supposed to take (or to be administered) over a specified period of time, and the individual doses that comprise the regimen are usually scheduled.

As used herein, the term “dosage form” refers to the form of pharmaceutical drug products with which they are marketed for use. Dosage forms are typically a mixture of active drug ingredients and inactive components (excipients), in a particular configuration (such as a capsule shell, for example), and apportioned into a particular dose. The dosage forms include tablets, capsules, troches, powders, solutions, suspensions, serums/gels, lotions, pasts, or the like, preferably in unit dosage forms suitable for simple administration of precise dosages.

As used herein, the terms “administer” and “administration,” when used with respect to a drug or an agent (including antibodies and antibiotics), means providing the drug or agent to a subject using any of the various methods or delivery systems for pharmaceutical compositions known to those skilled in the art. For example, the administration of the drug can be oral, nasal, parental, topical, ophthalmic, or transdermal delivery of the drug in the form of solid, semi-solid, lyophilized powder, or liquid dosage forms.

As used herein, the terms “co-administration” refers to the administration of a first active agent before, concurrently, or after the administration of a second active agent such that the biological effects of the two (or more) agents overlap.

As used herein, the term “parenteral route administration” refers to any route of drug administration other than oral (topical dosage forms are considered separately). The main parenteral routes of drug administration are intravenous (IV), intramuscular (IM), subcutaneous (SC), and intra-articular (IA), and the drug is usually administered using a hollow needle. The dosage forms for parenteral route injection are usually sterile solutions or suspensions of a drug in water or other physiologically suitable solvent. Administration volumes can range from milliliter to liter quantities. Parenteral dosage forms include injectable formulations (i.e., solutions, suspensions, emulsions, and dry powders for reconstitution immediately before injection), and administration route comprises intramammary infusions, intravaginal delivery systems, and implants. Injectable formulations must be sterile and free of pyrogens. The excipients included in parenteral solutions can include adjuncts in aseptic processing of products, inert gases, tonicity-adjusting agent to achieve isotonicity of the formulation, and other substances described above in “excipients”. (https://www.merckvetmanual.com/pharmacology/pharmacology-introduction/routes-of-administration-and-dosage-forms-of-drugs #v3329277)

As used herein, the term “topical route of administration” or “topical administration” refers to local application of therapeutic agents to the skin to control external and internal pathogens, including transdermal delivery route. Drugs or therapeutic agents for topical use include antibiotics, antiseptics, antimicrobials, anti-inflammatory agents, and skin emollients, which can be in the form of solids (powders), semisolids (creams, ointments, pastes, or gels (or serums)), and liquids (solutions, suspension concentrates, emulsions, emulsifiable concentrates, paints, and tinctures). Examples of t liquid solution include eye drops, ear drops, and lotions. A lotion is usually an aqueous solution (or emulsion) for application to inflamed, ulcerated skin. Gels and liquid serums are nongreasy, semisolid, aqueous solutions. comprising polymers to give continuous structure to the hydrophilic liquid solution. The polymers used for gel or serum can be natural gums such as tragacanth, pectin, and agar; semisynthetic materials such as methylcellulose, hydroxymethylcellulose, and carboxymethylcellulose; and synthetic polymers. Medicaments are generally well released from gels, which are easily washed off because of their water miscibility. (https://www.merckvetmanual.com/pharmacology/pharmacology-introduction/routes-of-administration-and-dosage-forms-of-drugs #v3329277) Liquid serums are generally less viscous than gels, and contain higher percentages of key ingredients, allowing for rapid absorption and deeper penetration. Gels are generally denser than serums but are still easily applied and absorbed. (https://www.revivalabs.com/whats-the-difference-between-a-serum-gel-cream-or-lotion)

As used herein, the term “nucleic acid” refers to any polyribonucleotide or polydeoxyribonucleotide that may be unmodified RNA or DNA or modified RNA or DNA. Thus, for instance, nucleic acid as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or double-stranded, or a mixture of single- and double-stranded regions.

As used herein, the term “vector” refers to a nucleic acid capable of transporting another nucleic acid to which it has been linked, usually a DNA molecule that is used as a vehicle to carry a particular foreign nucleic acid sequence—usually DNA—into a host/recipient cell where it can be replicated and/or expressed. The vector typically includes features to facilitate the manipulation of DNA as well as a genetic marker for their selective recognition. The most common vectors are DNA plasmids, viruses (e.g., adenovirus, vaccinia, retrovirus, baculovirus, etc.) and artificial chromosomes. Certain vectors are capable of autonomous replication in a host cell into which they are introduced. In general, expression vectors of utility in recombinant DNA techniques are often in the form of plasmids. In the present specification, “plasmid” and “vector” may be used interchangeably as the plasmid is the most commonly used form of vector. The selection of vectors and methods to construct them are commonly known to persons of ordinary skill in the art and are described in general technical references (see, in general, “Recombinant DNA Part D,” Methods in Enzymology, Vol. 153, Wu and Grossman, eds., Academic Press (1987)). Desirably, the vector comprises regulatory sequences, such as transcription and translation initiation and termination codons, which are specific to the type of host (e.g., bacterium, fungus, plant or animal) into which the vector is to be introduced, as appropriate and taking into consideration whether the vector is DNA or RNA. Preferably, the vector comprises regulatory sequences that are specific to the genus of the host. Most preferably, the vector comprises regulatory sequences that are specific to the species of the host.

As used herein, the term “polypeptide” and the term “protein” are used interchangeably to refer to a polymer of amino acid residues connected to each other by peptide bonds between the alpha-amino and carboxy groups of adjacent residues, including modified amino acids (e.g., phosphorylated, glycated, glycosylated, etc.) and amino acid analogs, regardless of its size or function. The terms “protein” and “polypeptide” as used herein refer to both large polypeptides and small peptides. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, “sequence identity” or “identity” in the context of two nucleic acid- or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

### Overview

Herein, monoclonal antibodies targeting surface proteins of A. baumannii were developed for the purpose of prophylactic and therapeutic treatment of A. baumannii infection in subjects in need thereof.

In order to identify target antigens for antibodies which would protect the host against A. baumannii infection, the antigens required for pathogenesis and virulence had to be identified. To that end, a clinically relevant A. baumannii strain, i.e., AB5075, a strain that causes severe clinical disease in a murine pulmonary model was used, which shows a high level of resistance to most clinically used antibiotics. AB5075 was originally isolated from a Wounded Warrior with osteomyelitis when a bone culture was performed. After screening clinical isolates, developing molecular tools to manipulate the genome, and using bioinformatics, over 20 surface target proteins were cloned, and among them BauA and OmpW2 were selected as targets for the development of monoclonal antibodies, which are required for iron metabolism of the bacteria and bacterial survival and expressed on the bacterial surface.

Outer membrane protein W (OmpW) is one of porin proteins of A. baumannii. Porins are beta barrel proteins in the outer membrane of Gram-negative bacteria and act as a pore, through which molecules can diffuse. Unlike other membrane transport proteins, porins are large enough to allow passive diffusion, i.e., they act as channels that are specific to different types of molecules, and thus play a pivotal role in the uptake of nutritional substances such as iron. (Catel-Ferreira M, Marti S, Guillon L, Jara L, Coadou G, Molle V, Bouffartigues E, Bou G, Shalk I, Jouenne T, Vila-Farrés X, Dé E. The outer membrane porin OmpW of Acinetobacter baumannii is involved in iron uptake and colistin binding. FEBS Lett. 2016 January; 590(2):224-31).

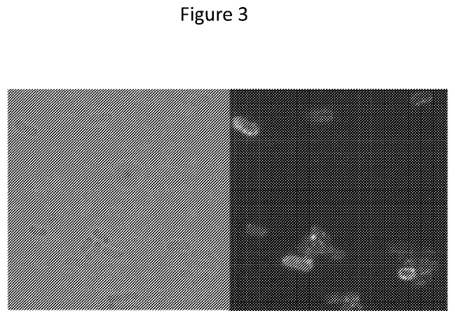

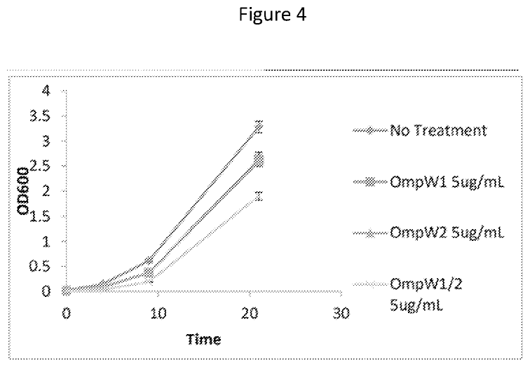

After sequencing the genome of AB5075, it was found that A. baumannii had three OmpW proteins (OmpW1, OmpW2, and OmpW3). OmpW3 was found to be the least important OmpW, and His-tagged versions of OmpW1 and OmpW2 (protein antigens) were constructed, which appeared to be more A. baumannii-unique (not found in other Gram-negative genomes), and polyclonal antibodies were generated. These polyclonal antibodies recognized the surface of A. baumannii in an immunofluorescence assay (FIG. 3) and reduced growth in media treated with an iron chelator (FIG. 4), showing OmpW1 and OmpW2 could be targeted with antibodies to have an effect on bacterial growth.

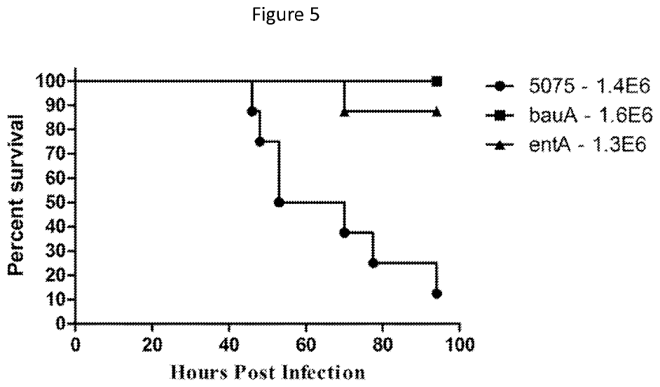

BauA is another protein expressed on the bacterial surface, which is a receptor protein on the outer membrane that binds to siderophore. Siderophore is a small molecule that binds and sequesters iron from the environment and carries it back to the bacteria, passing through bacterial outer membranes to the cytoplasm. There, iron can be used in a number of critical chemical reactions that keep the bacteria functioning, growing, and ultimately support pathogenic mechanisms to attack the host. Since like other forms of life, bacteria need iron and cannot grow unless they have a source of iron to draw from in the environment. The human body and its immune system actually restrict iron levels to keep bacterial growth in check; however, some pathogenic bacteria have found a way around the immune system by secreting siderophores. Using a BauA transposon mutants, it was shown that the gene encoding it was required for virulence (FIG. 5), demonstrating that BauA on the bacterial membrane is a good target for antibody development. BauA gene was cloned into a GST tagging plasmid vector, pGEX 6P-1.

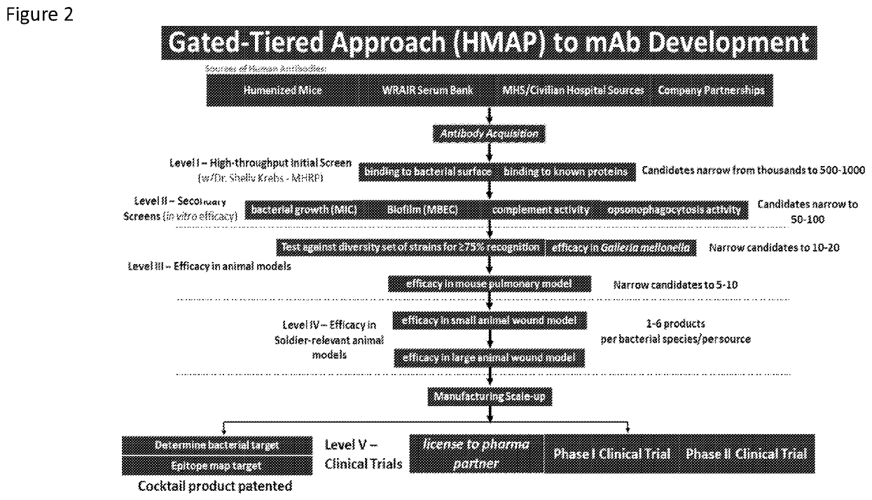

As illustrated in FIG. 2, cloned BauA and OmpW2 genes were overexpressed, and BauA and OmpW2 proteins were purified. Next, each protein was injected into mice to generate monoclonal antibodies using hybridoma cells. The supernatant of mouse B cell hybridoma library was screened and monoclonal antibodies against BauA (αBauA) and against OmpW2 (αOmpW2) were selected.

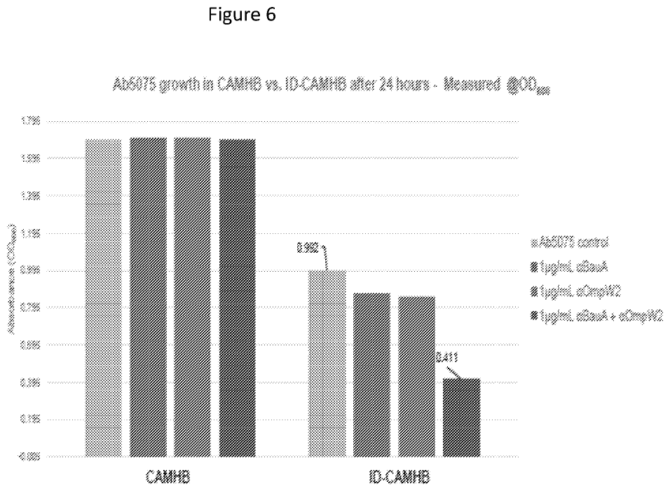

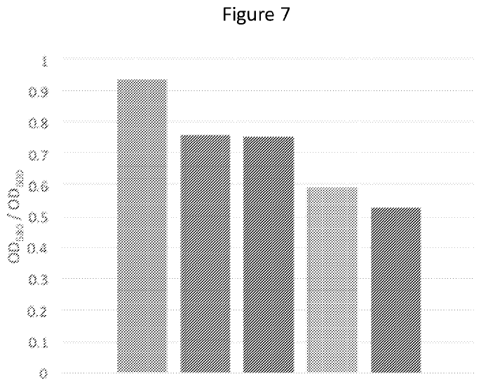

The effect of the purified antibodies (αBauA and αOmpW2) on growth of A. baumannii was tested and measured at OD₆₀₀. Each antibody, when provided alone into the bacterial culture media at 1.0 μg/mL reduced growth of AB5075 in iron-deficient culture media by ~10-15% compared with untreated bacterial culture. Growth was further reduced (up to 20%-30% growth inhibition), showing synergy effect when both antibodies were used in combination (FIG. 6). In nutrient-rich media, this effect was not apparent. In addition, when αBauA and/or αOmpW2 was added into the wells where biofilm could grow on pegs, antibody treatment prevented biofilm formation on the pegs when compared with samples untreated or treated with control αHIV antibody (FIG. 7). Addition of both αBauA and αOmpW2 also showed synergy antibiofilm effect (FIG. 7).

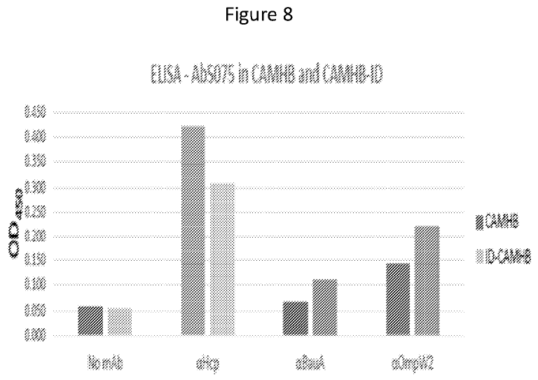

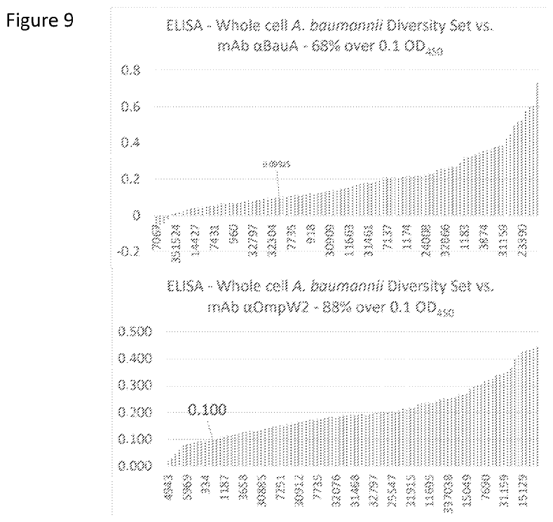

Next, it was demonstrated that low iron environment stimulated the bacteria to put more BauA and OmpW2 on its surface, by comparing AB5075 culture in nutrient-rich media and in iron-deficient media, which mimics physiological conditions (FIG. 8). This data suggests that when the bacteria is found in the host environment, it is actively putting BauA and OmpW2 to the outer membrane to capture more iron for survival. Next, it was demonstrated that αBauA and αOmpW2 recognized other strains of A. baumannii, and not just the model strain, AB5075 (FIG. 9), using a diversity set of A. baumannii generated by the Multidrug-Resistant Organism Repository Surveillance Network (MRSN).

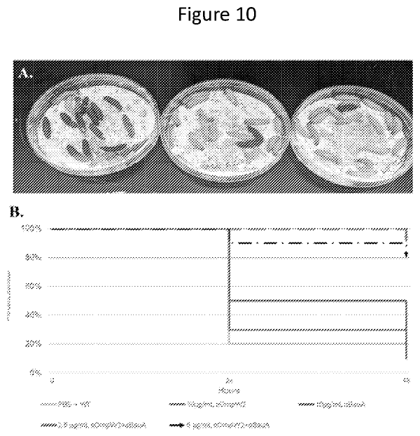

Next, the effect of the αBauA and αOmpW2 was tested in three in vivo animal models of infection; in Galleria mellonella, and in two murine models of infection. Galleria mellonella is a wax worm A. baumannii can kill in 24-48 hours (Jacobs et al. 2014). Worms were injected with each antibody or the combination of both αBauA and αOmpW2. One hour later, they were infected with 1.0×10⁶ colony forming units (CFU) of A. baumannii, and it is clearly shown that the combination of antibodies prevented infection and kept the worms alive (FIG. 10).

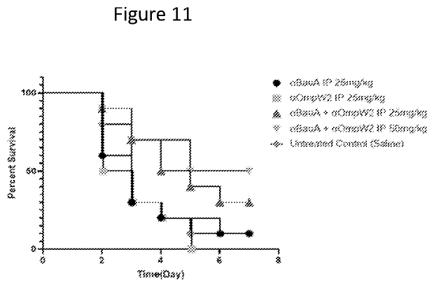

In the mouse pulmonary infection model, mice were injected with αBauA and/or αOmpW2 50 mg/kg via the intraperitoneal (i.p.) route, one day before the lungs of mice were inoculated with 5.0×10⁶ colony forming units (CFU) of bacteria through intranasal infection. In FIG. 11, Kaplan-Meier curves of representative murine pulmonary model experiment (n=10) showed that the combination of αBauA and αOmpW2 provided 70% protection on Day 4/5 when compared with single-treated or untreated mice and 50% protection over 8 days.

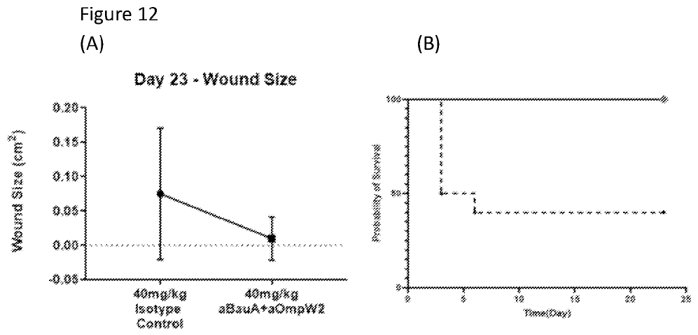

Lastly, it was demonstrated that the combination of both αBauA and αOmpW2 promoted wound healing in the murine wound model and provided 100% protection from sepsis and death. When the wound was treated with αBauA and αOmpW2 antibodies (40 mg/kg) prophylactically one day before infection, the wound size was dramatically reduced (>70%) on Day 23 (FIG. 12A). Separately, only the animals that received both αBauA and αOmpW2 survived 100%, whereas the animals that received the isotype-antibody (αOvalbumin antibody) survived only 40% (FIG. 12B).

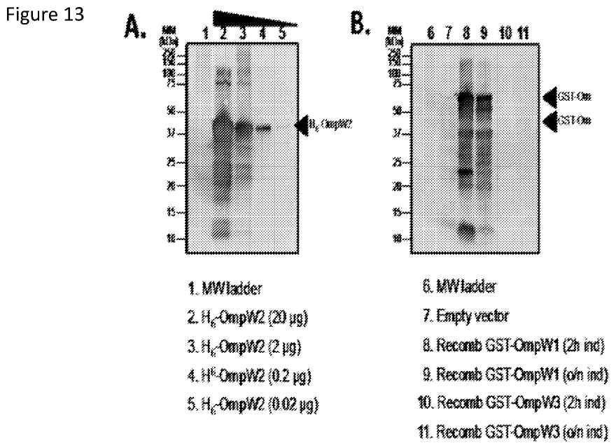

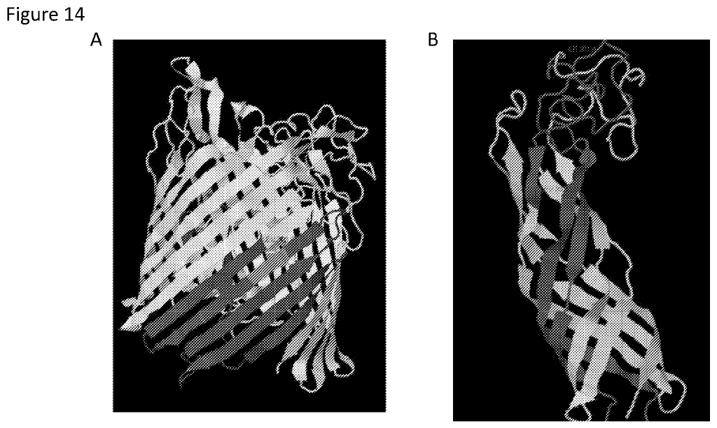

Further, epitope mapping identifies the area on the target protein that is responsible for antibody binding. It is possible that in the case of BauA, it may be a three dimensional epitope across multiple areas of the protein. For OmpW2, it was demonstrated here that the epitope is linear because on a Western blot, the antibody recognized the protein being denatured after SDS-PAGE electrophoresis (FIG. 13). Additionally, it was also found that the antibody recognized a recombinant version of OmpW1, and therefore the protective effect of the antibodies in this disclosure may be the result of recognizing and inhibiting both proteins, OmpW1 and OmpW2, which makes it more potent than an antibody that recognizes just one OmpW. The region of these epitopes (100 amino acids each) mapped to two surface-exposed regions of the proteins (FIG. 14).

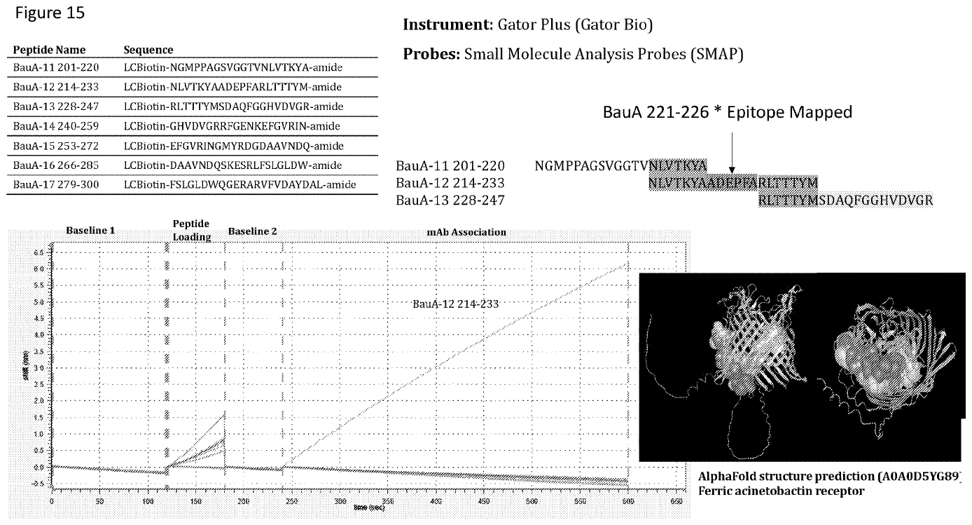

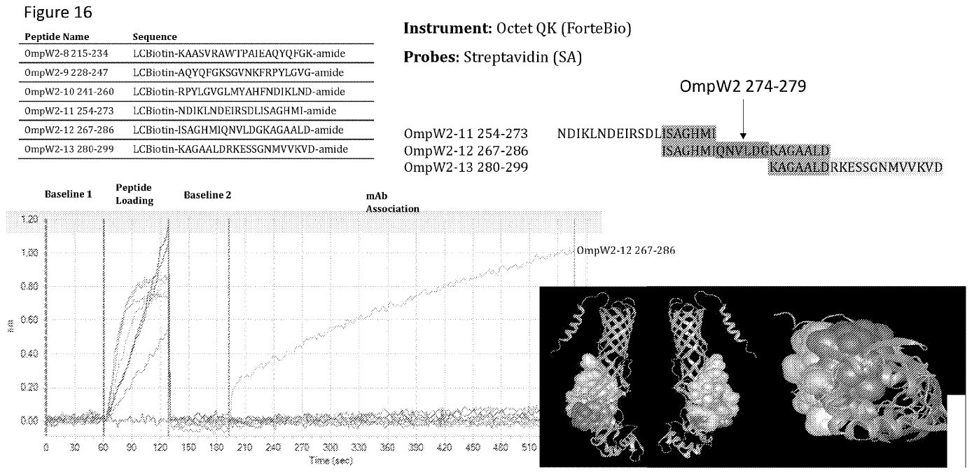

Additional epitope mapping studies using a Gator Plus instrument and 10 amino acid overlapping peptides to that 100 amino acid region of each protein identified the exact area (just 6 amino acids each) on the target protein that is responsible for antibody binding (FIG. 15 and FIG. 16).

In addition, the nucleic acid sequences and amino acid sequences in the heavy chain variable regions and light chain variable regions of αBauA and αOmpW2, including CDR1, 2, and 3 amino acid sequences are disclosed here.

Based on the results described above, it is one objective of the present disclosure to provide monoclonal antibodies against BauA and/or OmpW2 of Acinetobacter baumannii or antigen-binding fragments thereof for prophylactic or therapeutic treatment of A. baumannii infection in a mammalian subject, preferably human. By “prophylactic”, it is meant administered before infection, and by “therapeutic”, it is meant administered after infection.

The monoclonal antibodies provided in this disclosure are isolated mouse monoclonal antibodies, but other mammalian monoclonal antibodies can be contemplated, such as rat, rabbit, cow, pig, goat, sheep, donkey, horse, camelid, non-human primate, and human as well as chimeric monoclonal antibodies thereof and/or humanized monoclonal antibodies.

The monoclonal antibodies can be IgA, IgD, IgE, IgM, or IgG isotype, in particular IgG isotype, and they can be glycosylated forms of the antibodies, antibodies with enhanced stability, antibodies with enhanced specificity for BauA and OmpW2, antibodies with enhanced affinity for BauA and OmpW2.

The monoclonal antibodies against BauA or OmpW2 are heterotetramers comprising 2 heavy chains and 2 light chains, respectively. The monoclonal antibody against BauA comprises an amino acid sequence of SEQ ID NO:2 as heavy chain variable region and an amino acid sequence of SEQ ID NO:7 as light chain variable region. The monoclonal antibody against OmpW2 comprises an amino acid sequence of SEQ ID NO:12 or 22 as heavy chain variable region and an amino acid sequence of SEQ ID NO:17 or 27 as light chain variable region.

The monoclonal antibodies against BauA or OmpW2 as well as any antigen-binding fragments thereof comprise at least one complementarity determining region (CDR) (e.g., CDR1, CDR2 or CDR3 of the light chain variable region or heavy chain variable region) of a heavy- or light chain or a ligand binding portion thereof derived from the herein described mouse monoclonal antibodies, αBauA and αOmpW2, in combination with a heavy chain or light chain constant region, a framework region, or any portion thereof, that can be incorporated into an antibody of this disclosure. The antibody amino acid sequence can further optionally comprise at least one specified substitution, insertion or deletion as described herein or as known in the art.

The heavy chain variable region specified in SEQ ID NO:2 for an antibody against BauA (αBauA) comprises CDR1 defined by amino acid residues GYAFSNYW (SEQ ID NO:3), CDR2 IYPGDGDT (SEQ ID NO:4), and CDR3 ARGDFYYGSPFAY (SEQ ID NO:5), respectively. The light chain variable region specified in SEQ ID NO:7 for an antibody against BauA comprises CDR1 defined by amino acid residues KSLLHSNGNTY (SEQ ID NO:8), CDR2 RMS (SEQ ID NO:9), and CDR3 MQHLEYPLT (SEQ ID NO:10), respectively.

The heavy chain variable region of the monoclonal antibodies against BauA may comprise an amino acid sequence at least 90%, 92%, 95%, 97% 98%, 99% or more identical to the amino acid sequence of SEQ ID NO:2, and the heavy chain variable region CDRs may comprise an amino acid sequence at least 90%, 92%, 95%, 97% 98%, 99% or more identical to the amino acid sequence of SEQ ID NO:3, 4, or 5. The light chain variable region of the monoclonal antibodies against BauA may comprise an amino acid sequence at least 90%, 92%, 95%, 97% 98%, 99% or more identical to the amino acid sequence of SEQ ID NO:7, and the light chain variable region CDRs may comprise an amino acid sequence at least 90%, 92%, 95%, 97% 98%, 99% or more identical to the amino acid sequence of SEQ ID NO:8, 9, or 10.

The heavy chain variable region specified in SEQ ID NO:12 for an antibody against OmpW2 (αOmpW2) comprises CDR1 defined by amino acid residues GFNVKDYY (SEQ ID NO:13), CDR2 IDPENGKTI (SEQ ID NO:14), and CDR3 ATTWGVPY (SEQ ID NO:15), respectively. The light chain variable region specified in SEQ ID NO:17 for an antibody against OmpW2 comprises CDR1 defined by amino acid residues QSLLDSDGRTY (SEQ ID NO:18), CDR2 LVS (SEQ ID NO:19), and CDR3 WQGTHFPFT (SEQ ID NO:20), respectively.

In addition, the variable regions of another antibody against OmpW2 (αOmpW2-2) are provided. The heavy chain variable region specified in SEQ ID NO:22 for αOmpW2-2 comprises CDR1 defined by amino acid residues GFTFSNDW (SEQ ID NO:23), CDR2 IRLKSNNYAT (SEQ ID NO:24), and CDR3 TRPGNWYFDD (SEQ ID NO:25), respectively. The light chain variable region specified in SEQ ID NO:27 for αOmpW2-2 comprises CDR1 defined by amino acid residues QDINSY (SEQ ID NO:28), CDR2 RAN (SEQ ID NO:29), and CDR3 LQYDEFPLT (SEQ ID NO:30), respectively.

The heavy chain variable region of the monoclonal antibodies against OmpW2 may comprise an amino acid sequence at least 90%, 92%, 95%, 97% 98%, 99% or more identical to the amino acid sequence of SEQ ID NO:12 or 22, and the heavy chain variable region CDRs may comprise an amino acid sequence at least 90%, 92%, 95%, 97% 98%, 99% or more identical to the amino acid sequence of SEQ ID NO:13, 23, 14, 24, 25, or 15. The light chain variable region of the monoclonal antibodies against OmpW2 may comprise an amino acid sequence at least 90%, 92%, 95%, 97% 98%, 99% or more identical to the amino acid sequence of SEQ ID NO:17 or 27, and the light chain variable region CDRs may comprise an amino acid sequence at least 90%, 92%, 95%, 97% 98%, 99% or more identical to the amino acid sequence of SEQ ID NO:18, 28, 19, 29, 30, or 20.

The antigen-binding fragment can be a Fab fragment, Fab′ fragment, F(ab′)₂ fragment, Fv fragment, disulfide-stabilized Fv fragment (dsFv), single chain Fv fragment (scFv) and (scFv)₂, diabody, single domain antibody, nanobody, humabody or multispecific antibody formed from antigen-binding fragments.

In this disclosure, humanized monoclonal antibodies and human monoclonal antibodies against BauA or OmpW2 are suggested, which comprise light chains and heavy chains, each of the chains comprising at least part of a human constant region and framework region and at least part of a variable region derived from the mouse monoclonal antibodies, αBauA and αOmpW2, which bind to BauA and OmpW2 and inhibit or reduce A. baumannii infection in vitro and in vivo.

The antibodies and antigen-binding fragments thereof can also be formed by combining a Fab portion and an Fc region from different species, or by keeping the complementarity-determining regions and modifying the framework regions to that of another species.

Further, the antibodies and antigen-binding fragments thereof can be produced by any method, such as recombinant antibody construction, hybridoma technology, phage display, or any other methods known in the art, and they can be produced in any organisms or cell lines, including bacteria, yeast, insect, mammal or other type of cells or cell lines.

Another objective of the present disclosure is to provide isolated polynucleotides comprising nucleic acid sequences encoding the aforementioned monoclonal antibodies against BauA or OmpW2, comprising at least one specified sequence, domain, portion or variant thereof. In particular, the present disclosure provides nucleic acid sequences encoding the heavy chain variable region of anti-BauA comprising SEQ ID NO:1, the light chain variable region of anti-BauA comprising SEQ ID NO:6, the heavy chain variable region of anti-OmpW2 comprising SEQ ID NO:11 or 21, and the light chain variable region of anti-OmpW2 comprising SEQ ID NO:16 or 26. The polynucleotide may comprise a nucleic acid sequence at least 90%, 92%, 95%, 97% 98%, 99% or more identical to the nucleic acid sequence of SEQ ID NO:1, 6, 11, 21, 16, or 26.

The present disclosure further provides recombinant vectors comprising any of aforementioned nucleic acid sequences encoding the heavy chain variable region and/or light chain variable region of anti-BauA or a fragment thereof, and/or the heavy chain variable region and/or light chain variable region of anti-OmpW2 or a fragment thereof, recombinant vectors for gene amplification or protein expression encoded by those nucleic acid sequences, and host cells containing such nucleic acid sequences and/or recombinant vectors. The host cell can optionally be at least one selected from bacteria, yeast, insect, or mammalian cells such as COS, Vero, HeLa, NIH 3T3, HEK293, HEK293T, BHK, HUH7, HEPG2, HEP3B, U2OS, A549, HT1080, CAD, P19, L929, N2a, MCF-7, Y79, SO-Rb50, DUKX-X11, J558L, NS0, Sp2/0 cell, lymphoma cell, myeloma cell, B-cell/myeloma hybridoma cell, or any derivative, immortalized or transformed cell thereof.

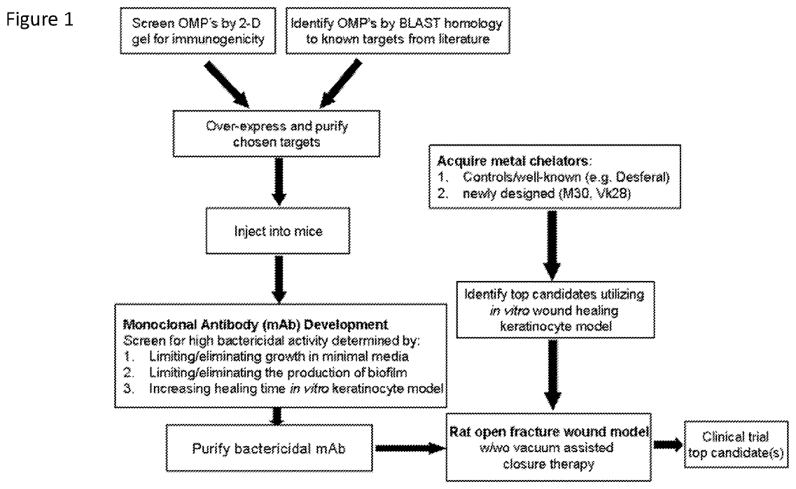

In one aspect, the present disclosure also provides the original conception (FIG. 1) and a method for producing monoclonal antibodies against BauA and OmpW2 (FIG. 2), comprising antigen screening and cloning, antigen protein purification, injection into a mouse, B-cell hybridoma formation, monoclonal antibody screening and cloning, and antibody purification, and testing in vitro and in vivo.

A variety of methods exist in the art for the production of monoclonal antibodies. For example, the monoclonal antibodies may be made by recombinant DNA methods, such as those described in U.S. Pat. No. 4,816,567. The DNA encoding the monoclonal antibodies of the invention can be readily isolated and sequenced using conventional procedures (e.g., by using oligonucleotide probes that are capable of binding specifically to genes encoding the heavy and light chains of murine antibodies, or such chains from human, humanized, or other sources). Once isolated, the DNA may be placed into expression vectors, which are then transformed into host cells that do not otherwise produce immunoglobulin protein, to obtain the synthesis of monoclonal antibodies in the recombinant host cells.

Another objective of the present disclosure is to provide a pharmaceutical composition comprising (a) at least one of isolated mouse monoclonal antibody against BauA or fragment thereof or mouse monoclonal antibody against OmpW2 or fragment thereof, and (b) a suitable carrier, excipient or diluent. The carrier, excipient or diluent can be pharmaceutically acceptable, according to known carriers, excipients or diluents. The composition can optionally further comprise at least one antibiotic or other antibodies that can be polyclonal or monoclonal antibodies, targeting other epitopes of A. baumannii BauA and/or OmpW2, or other surface proteins of A. baumannii or other ESKAPEE pathogens. The pharmaceutical composition can be a dosage form of solution, liquid serum, gel/hydrogel, lotion or lyophilized powder or granules, and administered through at least one administration route selected from parenteral(subcutaneous, intramuscular, intravenous, intrathecal) route or topical route.

Another objective of the present disclosure is to provide a method for administering a therapeutically effective amount to prevent or treat A. baumannii infection in a subject prior to, subsequent to, or during a related condition, as known in the art and/or as described herein, and this method can also be applied in order to prevent or treat other ESKAPEE pathogens. Since BauA and OmpW2 is conserved targets and are found in almost every strain of A. baumannii (FIG. 9), it is expected that the anti-BauA and OmpW2 antibodies described herein will be useful for treatment of infection by different strains of A. baumannii. Further, the method of using individual antibodies, or mixtures thereof targeting iron acquisition in this fashion for the prophylactic and therapeutic treatment of A. baumannii infections disclosed here can be applied to the prophylactic and therapeutic treatment of other ESKAPEE pathogens.

Another objective of the present disclosure is to provide a kit comprising the pharmaceutical compositions comprising at least one of isolated anti-BauA antibody or fragment thereof, or anti-OmpW2 antibody or fragment thereof. The pharmaceutical composition can be contained in a container, wherein the container can be optionally an auto-injector, a needle-free injector, patch, microneedle patch, spray can, squeezable pouch, plastic or metal container, containing a fixed volume of diluent or a physiologically acceptable buffer, e.g., sterilized saline or distilled water, to reconstitute the lyophilized powder or granules.

The kit can comprise antibiotics or other antibodies, polyclonal or monoclonal, against A. baumannii and/or other ESKAPEE pathogens, and the antibiotics and antibodies can be packaged with the anti-BauA and/or anti-OmpW2 monoclonal antibodies of this disclosure in the same container or separately packaged in another container in a different dosage form.

The kit may further comprise materials for field use of the invention of this disclosure, including non-flammable sterilized hand washing solution and wound washing solution, e.g., sterilized saline or distilled water, in a sprayable container, at least a pair of nitrile gloves, sterilized wound dressing materials, and information sheet describing antibody reconstituted method prior to administration.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### EXAMPLES

### Example 1. Materials and Methods

Bacterial growth conditions: Acinetobacter baumannii strain 5075 (AB5075) was cultured in either lysogeny broth (LB), CAMHB, or ID-CAMHB medium from an isolated colony, in the presence of 100 μg/mL ampicillin, and grown overnight at 37° C. shaken at 225 rpm. A 1:100 subculture was then grown in the same conditions until reaching mid-log phase, and cells were centrifuged (5 minutes, 5000×g), washed twice in 1× phosphate buffered saline (PBS), and resuspended in 1×PBS. Colony-forming units (CFU) enumeration was performed by spreading 10-fold serial dilutions of culture onto LB agar plates.

Enzyme-Linked Immunosorbent Assay (ELIZA): In addition to AB5075, a genetically diverse set of 100 A. baumannii strains was prepared as above. After bacterial growth, a final dilution corresponding to ~5.0×10⁵ CFU/well was seeded onto round-bottom 96-well plates and allowed to fully dry inside a Biosafety Cabinet (BSC). Next, either αBauA or αOmpW2 antibodies, or an isotype control were added to the wells and allowed to incubate at 37° C., then washed, followed by a horseradish peroxidase conjugated secondary antibody, and reading in a spectrophotometer at an absorbance of 450 nm.

Growth inhibition assay: Antibodies and controls were dispensed in round-bottom 96-well plates and later mixed with AB5075 cultures for a final cell density ≈1.0×10⁴ CFU/well. Then, static growth was measured at specific time points at an absorbance of 600 nm.

Galleria mellonella antibody treatment assay: Galleria mellonella waxworms were weighed out to a 200-300 mg range and used within two days of receipt from the vendor. After separating into groups according to planned experiment conditions, the larvae were placed onto petri dishes previously lined with filter paper. Thirty minutes before injecting, the petri dishes were stored in 4° C. so that larvae were stunned, allowing for easier handling. Using a glass syringe, larvae were injected into the top right proleg with 10 μL of either antibody, a combination of both, 1×PBS as a negative control. After incubating for ~1 hour at 37° C., an AB5075 culture ≈2.2×10⁵ CFUs was injected into bottom left proleg of each larva. All larvae were incubated at 37° C. and observed for survival over 48 hours.

Murine pulmonary model of infection: For this model, BALB/c mice of ~6 weeks of age were inoculated with either antibody, a combination of both, or an isotype control, at different doses on Day-1. On Day 0, they were infected with AB5075 by allowing the bacteria to be aspirated into the lungs. After infection, mice were weighed and observed for clinical signs daily over 8 days.

Murine wound model of infection: In this model, mice were treated similarly to the pulmonary model, but are infected by cutting an equally sized dorsal wound, and infecting the wound directly with AB5075. Observation and wound length measurement were carried out up to 23 days.

### Example 2. Preparation of Monoclonal Antibodies Against BauA and OmpW2 (αBauA and αOmpA)

First, the antigen protein (OmpW1) was cloned, and antibodies were purchased from AbD Serotec and tested in in vitro growth assays. Subsequently, assays showed that these antibodies bound to the surface of AB5075 (a model strain of Acinetobacter baumannii) using immunofluorescence (data not shown) and had a modest impact on reducing the growth of bacteria (data not shown).

Then, after sequencing the genome of AB5075, it was found that A. baumannii had three OmpW proteins (OmpW1, OmpW2, and OmpW3). The original monoclonals made to OmpW3 did not have enough efficacy in vitro to move into animal studies as they were made against to only one OmpW protein which is the least important OmpW. Therefore, to counter this, His-tagged versions of OmpW1 and OmpW2 (protein antigens) were constructed, which appeared to be more A. baumannii-unique (not found in other Gram-negative genomes), and polyclonal antibodies were generated as a cheaper alternative to study efficacy. These polyclonal antibodies also recognized the surface of A. baumannii in an immunofluorescence assay (FIG. 3) and reduced growth in media treated with an iron chelator (FIG. 4). This was preliminary data showing OMPs, and specifically OmpW1 and OmpW2, could be targeted with antibodies to have an effect on bacterial growth.

Then, other targets on the surface of A. baumannii were pursued, and transposon mutants of BauA of A. baumannii (U.S. Pat. No. 10,961,298 [application Ser. No. 15/505,375]) showed that the gene encoding it was required for virulence (FIG. 5). Thus, the bauA gene was cloned into pGEX 6P-1 and its protein was purified.

Next, mouse monoclonal antibodies (mAbs) against BauA and OmpW2 were made. Initially, these antibodies were made via fee for-service by Green Mountain Antibodies, Inc. (GMA). After screening the hybridomas, about 10 subclones were identified and stored (Tables 1-2)

It was aimed to make cocktails for both A. baumannii and Klebsiella pneumoniae using similar targets: fimbriae and siderophore receptors and do testing in animals. This was done for A. baumannii, but the K. pneumoniae part was not completed. However, confidence in this methods led to another study on a platform approach for antibody development named Human Monoclonal Antibody Platform (HMAP). HMAP was designed to take those previous targets and new targets and develop human mAbs (hu-mAbs) as opposed to murine mAbs. These hu-mAbs would be clinic-ready for testing in human patients.

For the selection of murine antibodies, the hybridoma subclones producing- and supernatants containing monoclonal antibodies against BauA and OmpW2 were tested for antibody activity and efficacy.

Supernatants from the original hybridomas were tested. They revealed that some of the subclones could produce antibodies binding to native A. baumannii when seeded onto ELISA plates (Table 3). From here, two subclones for anti-BauA antibody and anti-OmpW2 antibody were selected, 12C1 and 7C10 respectively (hereinafter αBauA and αOmpW2), which had the highest recorded binding to the native surface. These subclones were grown out to make purified antibodies and frozen back again with numerous aliquots so they could be thawed and more antibodies could be generated in the future. The purified antibody from these subclones were then used for all subsequent studies.

### Example 3. In Vitro Test of αBauA and αOmpW2

First, the effects on growth were reconfirmed with purified antibody. Each antibody alone when provided at 1.0 μg/mL had a slight effect on bacterial growth when measured by OD₆₀₀ and in low iron media (Cation-adjusted Müeller-Hinton Broth, which is typically used in MIC assays). When the antibodies were provided in combination, a synergy effect was observed (FIG. 6). This observation was recapitulated in biofilm data where if antibody was provided in wells where biofilm was grown on pegs, the antibody-treated wells prevented biofilm formation on the pegs when compared to isotype antibody controls (FIG. 7)

Further, because of this synergistic effect on growth in iron-depleted medium, it was examined if the low iron environment stimulated the bacteria to put more BauA and OmpW2 on its surface. AB5075 was grown to log phase, seeded, and dried on ELISA plates, and native ELISA was performed with αBauA (red), αOmpW2 (blue), and αHcp (green) antibodies. One hour after incubating the bacteria with antibodies, secondary antibody conjugated to HRP was added to each well for incubation. Developer was added and wells were measured in a plate reader at OD450. Indeed, when grown in this media and native ELISA was performed, both increased amounts of BauA and OmpW2 were found on the surface in low-iron media when compared with controls (FIG. 8). This data suggests that when the bacteria is found in the host environment, it is actively putting BauA and OmpW2 to the outer membrane to capture more iron for survival.

Lastly, it was tested if these antibodies recognized other strains of A. baumannii, and not just our model strain, AB5075. For this, a diversity set of A. baumannii were generated by the Multidrug-resistant Organism Repository Surveillance Network (MRSN) at WRAIR. The MRSN builds a diversity set of A. baumannii with 100 strains that share the core genome, but the remaining 5-10% of the genome is diverse for each strain (Galac et al. 2020). This set can be used to identify outliers where a drug won't work or if certain strains do not express or secrete target proteins to the surface, as was being evaluated here. Both the αBauA and αOmpW2 recognized the surface of the bacterium in a number of different strains (FIG. 9). αBauA recognized >68% of strains, and αOmpW2 recognized >88% strains. This is better data than a competing group that made antibodies to capsule that only recognized 38% of the strains they tested (Nielsen et al. 2021). The combination of these in vitro results informed us to take the next step into in vivo models of A. baumannii infection. Currently A. baumannii strains lacking BauA and OmpW1/OmpW2 are being tested if they are less virulent.

### Example 4. In Vivo Test of αBauA and αOmpW2

After sufficiently showing the antibodies had promise in vitro, it was tested the αBauA and αOmpW2 alone and in combination in three different animal models of infection.

The first was Galleria mellonella, which is a wax worm model, where A. baumannii can kill the wax worms in 24-48 hours (Jacobs et al. 2014). The worms were injected with either αBauA or αOmpW2 or the combination of both antibodies, then they were infected with 1.0×10⁶ colony forming units (CFU) of A. baumannii. FIG. 10A shows αBauA and αOmpW2 injection reduced dead larvae (differentiated by melanization, live (light) and dead larvae (dark)). FIG. 10B shows Kaplan-Meier survival curve over 48 hours evaluating G. mellonella infected with AB5075. Single mAb applications provide some protection, but the combination of both αBauA and αOmpW2 provides 90% and 80% protection compared with 0% survival of untreated controls, depending on the dose, 2.5 μg/mL and 5.0 μg/mL respectively.

These results were recapitulated in the mouse pulmonary model of infection (Jacobs et al. 2014). Mice were injected with αBauA and αOmpW2 50 mg/kg on Day −1 via the intraperitoneal (i.p.) route, the day before the lungs of mice were inoculated with an intranasal delivery of 5.0×10⁶ colony forming units (CFU) of bacteria. These experiments were repeated three times with n=10 animals for each biological replicate (FIG. 11). As one can see, mice receiving the antibody combination yielded 70% protection when all but one of the control animals died on Day 4. This is important because again our competitor who made antibody to capsule only saw 50% protection in a similar model (Nielsen et al. 2021).

Finally, the last murine model of infection that was used to evaluate our antibodies is the wound infection model (Thompson et al. 2014). In this model, a 6 mm punch biopsy is made on the dorsum of the mouse. A bacterial inoculum of 5.0×10⁴ CFU is applied to the wound and infection is monitored for 23+ days. Antibodies were injected the Day before i.p., and wounds were measured every other day for 23 days using an Aranz instrument that uses two lasers to calculate wound area. Separately, animals were monitored for survival. Unfortunately, some animals that were treated with the isotype control antibody (αOvalbumin) became sick, suffering from sepsis and perished. In contrast, αBauA and αOmpW2 antibody combination protected the mice from sepsis and death with 100% survival with mice that were treated (FIG. 12). These results were unexpected because of 70% protection in the pulmonary model, but that model uses a larger inoculum, and the bacteria have more direct access to the bloodstream, where sepsis and degradation of other organs would occur more quickly. In real world settings, smaller inoculums are the norm, and therefore, the antibodies may perform better with respect to human patient outcomes.

### Example 5. Epitope Mapping and Antibody Sequences

Epitope mapping identifies the area on the target protein that is responsible for antibody binding. Cloning of larger 100 amino acid segments of BauA and OmpW2 allowed us to identify specific 100 amino acid regions of each protein that bind to each antibody. This was then confirmed using 20 amino acid overlapping peptides that identified a 6 amino acid epitope for each protein using the Gator Plus Instrument (FIG. 15-16).

For OmpW2, the epitope is linear because on a Western blot, it recognized the protein being denatured after SDS-PAGE electrophoresis. Additionally, it was found that the antibody recognized a recombinant version of OmpW1, and therefore the antibody's protective phenotypes may be the result of recognizing and inhibiting both proteins making it more potent than antibody that recognized just one OmpW (FIG. 13). Unfortunately, the αOmpW2 did not recognize recombinant OmpW3 as well, but this protein is only 60% homologous to OmpW1 and OmpW2 and more closely resembles OmpW from E. coli or OprG from Pseudomonas aeruginosa.

The other important piece of this disclosure is the sequence of each antibody. Green Mountain Antibodies, Inc. sequenced αBauA and αOmpW2. Those results are presented in the Appendix section below, and a separate report in a .pdf format can be provided. This sequence then becomes the basis for future recombinant antibody constructs.

The αBauA and αOmpW2 antibodies were sequenced and those sequences are listed below.

αBauA-Antibody Sequence
Heavy Chain
DNA Sequence
gaggtgcagctgcaggagtctggggctgacctggtgaggcctgggtcctcagtgaagatttcctgcaaggcttctggctatgcattcagtaactactg
gatgcactggatgaagcagaggcctggacagggtcttgagtggattggacagatttatcctggagatggtgatactaactacaatggaaagttcaag
ggtaaagccacactgactgcagacaaatcctccagctcagcctacatgcagctcagcagcctaatatctgaggactctgcggtctatttctgtgcaag
aggggatttttactacggtagcccgtttgcttactggggccaagggactctggtcactgtctctgcag
Predicted Protein Sequence
Complementarity determining regions (CDRs) are underlined.
EVQLQESGADLVRPGSSVKISCKASGYAFSNYWMHWMKQRPGQGLEWIGQIYPGDGDTNYNGKFKGK
ATLTADKSSSSAYMQLSSLISEDSAVYFCARGDFYYGSPFAYWGQGTLVTVSA
Light Chain
DNA Sequence
gatattgtgatgactcaggctgcaccctctgtacctgtcactcctggagagtcagtatccatctcctgcaggtctagtaagagtctcctgcatagtaa
tggcaacacttacttgtattggttcctgcagaggccaggccagtctcctcagctcctgatttatcggatgtccaaccttgcctcaggagtcccagaca
ggttcagtggcagtgggtcaggaactgctttcacactgagaatcagtagagtggaggctgaggatgtgggtgtttattactgtatgcaacatctagaa
tatccgctcacgttcggtgctgggaccaagctggagctgaaac
Predicted Protein Sequence
Complementarity determining regions (CDRs) are underlined.
DIVMTQAAPSVPVTPGESVSISCRSSKSLLHSNGNTYLYWFLQRPGQSPQLLIYRMSNLASGVPDRFSGS
GSGTAFTLRISRVEAEDVGVYYCMQHLEYPLTFGAGTKLELK
αOmpW2-Antibody Sequence
Heavy Chain
DNA Sequence
gaggtgcagctgcaggagtctggggctgaacttgtgaggccaggggccttaattaagttgtcctgcaaagcttctggcttcaatgttaaagactacta
tatgcaatgggtgaagcagaggcctgaacagggcctggagtggattggatggattgatcctgagaatgggaaaactatatgtgacccgaagttcca
gggcaaggccagtataacagcagacacatcctccaacacagcctacctgcacctcagcagcctgacatctgaggacactgccgtctattactgtg
ctactacatggggggttccttactggggccaagggactctggtcactgtccgcag
Predicted Protein Sequence
Complementarity determining regions (CDRs) are underlined.
EVQLQESGAELVRPGALIKLSCKASGFNYKDYYMQWVKQRPEQGLEWIGWIDPENGKTICDPKFQGKAS
ITADTSSNTAYLHLSSLTSEDTAVYYCATTWGVPYWGQGTLVTVSA
Light Chain
DNA Sequence
gatgttgtgatgacccagactccactcactttgccggttaccattggacaaccagcctccatctcttgcaagtcaagtcagagcctcttagatagtga
tgggaggacatatttgaattggttgtcacagaggccaggccagtctccaaagcgcctaatctatctggtgtctaaactggactctggagtccctgaca
ggttcactggcagtggatcagggacagatttcacactgaaaatcagcagagtggaggctgaggatttgggagtttattattgctggcaaggtacacac
tttccattcacgttcggctcggggacaaagttggaaataaaac
Predicted Protein Sequence
Complementarity determining regions (CDRs) are underlined.
DVVMTQTPLTLPVTIGQPASISCKSSQSLLDSQGRTYLNWLSQRPGQSPKRLIYLVSKLDSGVPDRFTGS
GSGTDFTLKISRVEAEDLGVYYCWQGTHFPFTFGSGTKLEIK
αOmpW2-2-Antibody Sequence
Heavy Chain 2
DNA Sequence
gaggtgcagctgcaggagtctggaggaggcttggtacaacctggaggatccatgaaactctcctgtattgccgctggattcactttcagtaacgactg
gatgaactgggtccgccagtctccagagaaggggcttgagtgggttgctgaaattagattgaaatctaataattatgcaacacgttatgtggagtctg
tgaaagggaggttcaccatctcaagagatgattccaaaagttctgtctacctgcaaatgaacaacttaagagctgaagacactggcatttattactgt
accaggccgggtaactggtacttcgatgactggggcgcagggaccacggtcaccgtctcctcag
Predicted Protein Sequence
Complementarity determining regions (CDRs) are underlined.
EVQLQESGGGLVQPGGSMKLSCIASGFTFSNDWMNWVRQSPEKGLEWVAEIRLKSNNYATRTVESVKGRFTISR
DDSKSSVYLQMNNLRAEDTGIYYCTRPGNWYFDDWGAGTTVTVSS
Light Chain
DNA Sequence
gacatcaaggtgacccagtctccatcttccatgtatgcatctcttggagagagagtcactatcacttgcaaggcgagtcaggacattaatagctattt
aagctggttccagcagaaaccagggaaatctcctaagaccctgatctatcgtgcaaacagattggtagatggggtcccatcaggttcagtggcagtgg
atctgggcaagattattctctcaccatcagcagcctggagtatgaagatttgggaatttattattgtctacagtatgatgagtttccgctcacgttcg
gtgctgggaccaagctggagctgaaac
Predicted Protein Sequence
Complementarity determining regions (CDRs) are underlined.
DIKVTQSPSSMYASLGERTITCKASQDINSYLSWFQQKPGKSPKTLIYRANRLVDGVPSRFSGSGSGQDYSLTISS
LEYEDLGIYYCLQYDEFPLTFGAGTKLELK

TABLE 4
Nucleotide and amino acid sequences of aBauA and «OmpW2

| SEQ | | | | |
|---|---|---|---|---|
| ID | | | | |
| NO | Antibody | | | Sequence |
| 1 | αBauA | Heavy | DNA seq. | gaggtgcagctgcaggagtctggggctgacctggtgaggcctgggtcctcagtgaag |
| | | | | atttcctgcaaggcttctggctatgcattcagtaactactggatgcactggatgaagca |
| | | | | gaggcctggacagggtcttgagtggattggacagatttatcctggagatggtgatact |
| | | | | aactacaatggaaagttcaagggtaaagccacactgactgcagacaaatcctccag |
| | | | | ctcagcctacatgcagctcagcagcctaatatctgaggactctgcggtctatttctgtg |
| | | | | caagaggggatttttactacggtagcccgtttgcttactggggccaagggactctggtc |
| | | | | actgtctctgcag |
| 2 | | Chain | a.a. seq. | EVQLQESGADLVRPGSSVKISCKASGYAFSNYWMHWMKQRPGQ |
| | | | | GLEWIGQIYPGDGDTNYNGKFKGKATLTADKSSSSAYMQLSSLISED |
| | | | | SAVYFCARGDFYYGSPFAYWGQGTLVTVSA |
| 3 | | | CDR1 | GYAFSNYW |
| | | | (a.a.) | |
| 4 | | | CDR2 | IYPGDGDT |
| | | | (a.a.) | |
| 5 | | | CDR3 | ARGDFYYGSPFAY |
| | | | (a.a.) | |
| 6 | | Light | DNA seq. | gatattgtgatgactcaggctgcaccctctgtacctgtcactcctggagagtcagtatc |
| | | Chain | | catctcctgcaggtctagtaagagtctcctgcatagtaatggcaacacttacttgtattg |
| | | | | gttcctgcagaggccaggccagtctcctcagctcctgatttatcggatgtccaaccttg |
| | | | | cctcaggagtcccagacaggttcagtggcagtgggtcaggaactgctttcacactgag |
| | | | | aatcagtagagtggaggctgaggatgtgggtgtttattactgtatgcaacatctagaa |
| | | | | tatccg ctcacgttcggtgctgggaccaagctggagctgaaac |
| 7 | | | a.a. seq. | DIVMTQAAPSVPVTPGESVSISCRSSKSLLHSNGNTYLYWFLQRPG |
| | | | | QSPQLLIYRMSNLASGVPDRFSGSGSGTAFTLRISRVEAEDVGVYYC |
| | | | | MQHLEYPLTFGAGTKLELK |
| 8 | | | CDR1 | KSLLHSNGNTY |
| | | | (a.a.) | |
| 9 | | | CDR2 | RMS |
| | | | (a.a.) | |
| 10 | | | CDR3 | MQHLEYPLT |
| | | | (a.a.) | |
| 11 | αOmp | Heavy | DNA seq. | Gaggtgcagctgcaggagtctggggctgaacttgtgaggccaggggccttaattaag |
| | | | | ttgtcctgcaaagcttctggcttcaatgttaaagactactatatgcaatgggtgaagca |
| | | | | gaggcctgaacagggcctggagtggattggatggattgatcctgagaatgggaaaa |
| | | | | ctatatgtgacccgaagttccagggcaaggccagtataacagcagacacatcctcca |
| | | | | acacagcctacctgcacctcagcagcctgacatctgaggacactgccgtctattactg |
| | | | | tgctactacatggggggttccttactggggccaagggactctggtcactgtctccgca |
| | | | | g |
| 12 | W2 | Chain | a.a. seq. | EVQLQESGAELVRPGALIKLSCKASGFNVKDYYMQWVKQRPEQGL |
| | | | | EWIGWIDPENGKTICDPKFQGKASITADTSSNTAYLHLSSLTSEDTAV |
| | | | | YYCATTWGVPYWGQGTLVTVSA |
| 13 | | | CDR1 | GFNVKDYY |
| | | | (a.a.) | |
| 14 | | | CDR2 | IDPENGKTI |
| | | | (a.a.) | |
| 15 | | | CDR3 | ATTWGVPY |
| | | | (a.a.) | |
| 16 | | Light | DNA seq. | gatgttgtgatgacccagactccactcactttgccggttaccattggacaaccagcctc |
| | | | | catctcttgcaagtcaagtcagagcctcttagatagtgatgggaggacatatttgaatt |
| | | | | ggttgtcacagaggccaggccagtctccaaagcgcctaatctatctggtgtctaaact |
| | | | | ggactctggagtccctgacaggttcactggcagtggatcagggacagatttcacactg |
| | | | | aaaatcagcagagtggaggctgaggatttgggagtttattattgctggcaaggtacac |
| | | | | actttccattcacgttcggctcggggacaaagttggaaataaaac |
| 17 | | Chain | a.a. seq. | DVVMTQTPLTLPVTIGQPASISCKSSQSLLDSDGRTYLNWLSQRPG |
| | | | | QSPKRLIYLVSKLDSGVPDRFTGSGSGTDFTLKISRVEAEDLGVYYCW |
| | | | | QGTHFPFTFGSGTKLEIK |
| 18 | | | CDR1 | QSLLDSDGRTY |
| | | | (a.a.) | |
| 19 | | | CDR2 | LVS |
| | | | (a.a.) | |
| 20 | | | CDR3 | WQGTHFPFT |
| | | | (a.a.) | |
| 21 | αOmp | Heavy | DNA seq. | [symbol] |
| 22 | W2-2 | Chain | a.a. seq. | [symbol] |
| 23 | | | CDR1 | GFTFSNDW |
| | | | (a.a.) | |
| 24 | | | CDR2 | IRLKSNNYAT |
| | | | (a.a.) | |
| 25 | | | CDR3 | TRPGNWYFDD |
| | | | (a.a.) | |
| 26 | | Light | DNA seq. | [symbol] |
| 27 | | Chain | a.a. seq. | [symbol] |
| 28 | | | CDR1 | QDINSY |
| | | | (a.a.) | |
| 29 | | | CDR2 | RAN |
| | | | (a.a.) | |
| 30 | | | CDR3 | LQYDEFPLT |
| | | | (a.a.) | |

| | | |
|---|---|---|
| 31 | BauA epitope (a.a.) | ADEPFA |
| 32 | OmpW2 epitope (a.a.) | QNVLDG |

[symbol]

indicates data missing or illegible when filed

TABLE 5
purified antibody samples and secondary reagents

| Antibody Code | Antigen | Amount | |
|---|---|---|---|
| AbD16330.1 | His-OmpW | 250 | μg |
| AbD16331.1 | His-OmpW | 250 | μg |
| AbD16332.1 | His-OmpW | 250 | μg |
| AbD16334.1 | His-OmpW | 250 | μg |
| AbD16335.1 | His-OmpW | 250 | μg |
| AbD16336.1 | His-OmpW | 250 | μg |
| AbD16337.1 | His-OmpW | 250 | μg |
| AbD16338.1 | His-OmpW | 250 | μg |
| AbD16340.1 | His-OmpW | 250 | μg |
| AbD16343 1 | His-OmpW | 250 | μg |
| AbD16345.1 | His-OmpW | 2 × 210 | μg |
| AbD16347.1 | His-OmpW | 2 × 230 | μg |
| AbD16348.1 | His-OmpW | 250 | μg |
| AbD16350.1 | His-OmpW | 250 | μg |

Secondary antibodies

| | | | |
|---|---|---|---|
| 0500-0099-sample | anti-Human Fab | 20 | μl |
| (=STAR126P) | HRP conjugated | | |
| 0500-0100-sample | anti-Human Fab | 20 | μl |
| (=STAR126A) | AP conjugated | | |

All antibodies are from a large-scale expression and are in 3×PBS buffer. The samples are stored at −20° C. or below for long-term storage. When thawed, it is recommended to aliquot the sample as needed, keep one aliquot at 4° C. and store the remaining aliquots at −20° C. or below. The thawed sample should be kept at 4° C. not longer than a few days, since azide was no added to the buffer. The antibodies are all obtained from our HuCAL® library* after panning against the antigen indicated in the table. The purified antibodies have been tested in ELISA.

### CONCLUSIONS

Overall, it was postulate that the combination of αBauA and αOmpW2 disrupts iron acquisition, which negatively impacts growth and bacterial functions in vivo leading to the clearance of bacteria and prevention of infection. It is hypothesize that the addition of a third antibody to this cocktail could further enhance protection and efficacy, and antibody development against other protein targets that are required for virulence is being explored.

### REFERENCES

1. Gallagher L A, Ramage E, Weiss E J, Radey M, Hayden H S, Held K G, Huse H K, Zurawski D V, Brittnacher M J, Manoil C. (2015) Resources for Genetic and Genomic Analysis of Emerging Pathogen Acinetobacter baumannii. J Bacteriol. 2015 Jun. 15; 197(12):2027-35.
2. Galac M R, Snesrud E, Lebreton F, Stam J, Julius M, Ong A C, Maybank R, Jones A R, Kwak Y I, Hinkle K, Waterman P E, Lesho E P, Bennett J W, Mc Gann P. (2020) A Diverse Panel of Clinical Acinetobacter baumannii for Research and Development. Antimicrob Agents Chemother. 2020 Sep. 21; 64(10):e00840-20.
3. Nielsen T B, Yan J, Slarve M, Lu P, Li R, Ruiz J, Lee B, Burk E, Talyansky Y, Oelschlaeger P, Hurth K, Win W, Luna B M, Bonomo R A, Spellberg B. Monoclonal Antibody Therapy against Acinetobacter baumannii. Infect Immun. 2021 Sep. 16; 89(10):e001622.
4. Jacobs A C, Thompson M G, Black C C, Kessler J L, Clark L P, McQueary C N, Gancz H Y, Corey B W, Moon J K, Si Y, Owen M T, Hallock J D, Kwak Y I, Summers A, Li C Z, Rasko D A, Penwell W F, Honnold C L, Wise M C, Waterman P E, Lesho E P, Stewart R L, Actis L A, Palys T J, Craft D W, Zurawski D V. AB5075, a Highly Virulent Isolate of Acinetobacter baumannii, as a Model Strain for the Evaluation of Pathogenesis and Antimicrobial Treatments. mBio. 2014 May 27; 5(3):e01076-14. doi: 10.1128/mBio.01076-14.
5. Thompson M G, Black C C, Pavlicek R L, Honnold C L, Wise M C, Alamneh Y A, Moon J K, Kessler J L, Si Y, Williams R, Yildirim S, Kirkup B C Jr, Green R K, Hall E R, Palys T J, Zurawski D V. (2014) Validation of a novel murine wound model of Acinetobacter baumannii infection. Antimicrob Agents Chemother. 2014; 58(3):1332-42.

1. A monoclonal antibody against BauA of Acinetobacter baumannii or an antigen-binding fragment thereof, comprising;
(a) a heavy chain variable domain comprising an amino acid sequence of SEQ ID NO:2, or an amino acid sequence at least 90% identical to the SEQ ID NO:2, and/or
(b) a light chain variable domain comprising an amino acid sequence of SEQ ID NO:7, or an amino acid sequence at least 90% identical to the SEQ ID NO:7.

2. The monoclonal antibody against BauA of A. baumannii or an antigen-binding fragment thereof of claim 1, wherein
the heavy chain variable domain comprises at least one of CDR1 of an amino acid sequence of SEQ ID NO:3 (GYAFSNYW), CDR2 of an amino acid sequence of SEQ ID NO:4 (IYPGDGDT), CDR3 of an amino acid sequence of SEQ ID NO:5 (ARGDFYYGSPFAY), and/or an amino acid sequence at least 90% identical to the SEQ ID NO:3, 4, or 5, and
the light chain variable domain comprises at least one of CDR1 of an amino acid sequence of SEQ ID NO:8 (KSLLHSNGNTY), CDR2 of an amino acid sequence of SEQ ID NO:9 (RMS), CDR3 of an amino acid sequence of SEQ ID NO:10 (MQHLEYPLT), and/or an amino acid sequence at least 90% identical to the SEQ ID NO:8, 9, or 10.

3. The monoclonal antibody against BauA of A. baumannii or an antigen-binding fragment thereof of claim 1, wherein the monoclonal antibody is a mammal (such as mouse, rat, rabbit, cow, pig, goat, sheep, donkey, horse, camelid, non-human primate, or human) antibody or chimeric antibody thereof, in particular mouse antibody, human antibody, or chimeric antibody thereof, and wherein, optionally, the antibody comprises a subtype of heavy chain selected from IgG1 or a variant thereof, IgG2 or a variant thereof, IgG3 or a variant thereof, IgG4 or a variant thereof, IgA or a variant thereof, IgE or a variant thereof, IgM or a variant thereof, or IgD or a variant thereof.

4. (canceled)

5. The monoclonal antibody against BauA of A. baumannii or an antigen-binding fragment thereof of claim 1, wherein the antibody is or comprises mouse IgG or human IgG.

6. The monoclonal antibody against BauA of A. baumannii or an antigen-binding fragment thereof of claim 1, (i) wherein the antibody or an antigen-binding fragment thereof comprises at least one amino acid sequence selected from SEQ ID NO:3, 4, 5, 8, 9, 10, or an amino acid sequence at least 90% identical to the SEQ ID NO:3, 4, 5, 8, 9, or 10, (ii) wherein the antibody is a humanized antibody comprising at least one amino acid sequence selected from SEQ ID NO:3, 4, 5, 8, 9, 10, or an amino acid sequence at least 90% identical to the SEQ ID NO:3, 4, 5, 8, 9, or 10, and/or (iii) wherein the antigen-binding fragment is a Fab fragment, Fab′ fragment, F(ab′)₂ fragment, Fv fragment, disulfide-stabilized Fv fragment (dsFv), single chain Fv fragment (scFv) and (scFv)₂, diabody, single domain antibody, nanobody, humabody or multispecific antibody formed from antigen-binding fragments.

7. (canceled)

8. (canceled)

9. A monoclonal antibody against OmpW2 of Acinetobacter baumannii or an antigen-binding fragment thereof, comprising;
(c) a heavy chain variable domain comprising an amino acid sequence of SEQ ID NO: 12 or 22, or an amino acid sequence at least 90% identical to the SEQ ID NO:12 or 22, and/or
(d) a light chain variable domain comprising an amino acid sequence of SEQ ID NO: 17 or 27, or an amino acid sequence at least 90% identical to the SEQ ID NO: 17 or 27.

10. The monoclonal antibody against OmpW2 of A. baumannii or an antigen-binding fragment thereof of claim 9, wherein
the heavy chain variable domain comprises at least one of CDR1 of an amino acid sequence of SEQ ID NO:13 (GFNVKDYY) or SEQ ID NO:23 (GFTFSNDW), CDR2 of an amino acid sequence of SEQ ID NO:14 (IDPENGKTI) or SEQ ID NO:24 (IRLKSNNYAT), CDR3 of an amino acid sequence of SEQ ID NO: 15 (ATTWGVPY) or SEQ ID NO:25 (TRPGNWYFDD), and/or an amino acid sequence at least 90% identical to the SEQ ID NO:13, 23, 14, 24, 25 or 15, and
the light chain variable domain comprises at least one of CDR1 of an amino acid sequence of SEQ ID NO:18 (QSLLDSDGRTY) or SEQ ID NO:28 (QDINSY), CDR2 of an amino acid sequence of SEQ ID NO:19 (LVS) or SEQ ID NO: 29 (RAN), CDR3 of an amino acid sequence of SEQ ID NO:20 (WQGTHFPFT) or SEQ ID NO:30 (LQYDEFPLT), and/or an amino acid sequence at least 90% identical to the SEQ ID NO:18, 28, 19, 29, 30 or 20.

11. The monoclonal antibody against OmpW2 of A. baumannii or an antigen-binding fragment thereof of claim 9, (i) wherein the monoclonal antibody is a mammal (such as mouse, rat, rabbit, cow, pig, goat, sheep, donkey, horse, camelid, non-human primate, or human) antibody or chimeric antibody thereof, in particular mouse antibody, human antibody, or chimeric antibody thereof; (ii) wherein the antibody comprises a subtype of heavy chain selected from IgG1 or a variant thereof, IgG2 or a variant thereof, IgG3 or a variant thereof, IgG4 or a variant thereof, IgA or a variant thereof, IgE or a variant thereof, IgM or a variant thereof, of IgD or a variant thereof, and/or (iii) wherein the antibody is or comprises mouse IgG or human IgG; and/or.

12. (canceled)

13. (canceled)

14. The monoclonal antibody against OmpW2 of A. baumannii or an antigen-binding fragment thereof of claim 9, wherein the antibody or an antigen-binding fragment thereof comprises at least one amino acid sequence selected from SEQ ID NO:13, 23, 14, 24, 15, 25, 18, 28, 19, 29, 20, 30 or an amino acid sequence at least 90% identical to the SEQ ID NO:13, 23, 14, 24, 15, 25, 18, 28, 19, 29, 30 or 20.

15. The monoclonal antibody against OmpW2 of A. baumannii or an antigen-binding fragment thereof of claim 9, wherein the antibody is a humanized antibody comprising at least one amino acid sequence selected from SEQ ID NO:13, 23, 14, 24, 15, 25, 18, 28, 19, 29, 20, 30 or an amino acid sequence at least 90% identical to the SEQ ID NO:13, 23, 14, 24, 15, 25, 18, 28, 19, 29, 30 or 20.

16. The monoclonal antibody against OmpW2 of A. baumannii or an antigen-binding fragment thereof of claim 9, wherein the antigen-binding fragment is a Fab fragment, Fab′ fragment, F(ab′)₂ fragment, Fv fragment, disulfide-stabilized Fv fragment (dsFv), single chain Fv fragment (scFv) and (scFv)₂, diabody, single domain antibody, nanobody, humabody or multispecific antibody formed from antigen-binding fragments.

17. (canceled)

18. (canceled)

19. (canceled)

20. (canceled)

21. (canceled)

22. A host cell comprising at least one nucleic acid molecule selected from SEQ ID NO:1, 6, 11, 21, 16, 26, or a nucleic acid sequence at least 90% identical to the SEQ ID NO:1, 6, 11, 21, 26 or 16, and/or a recombinant vector comprising at least one nucleic acid molecule selected from SEQ ID NO:1, 6, 11, 21, 16, 26, or a nucleic acid sequence at least 90% identical to the SEQ ID NO:1, 6, 11, 21, 26, or 16.

23. (canceled)

24. (canceled)

25. A composition for prophylactic or therapeutic treatment of A. baumannii infection, comprising a monoclonal antibody against BauA of Acinetobacter baumannii or an antigen-binding fragment thereof, comprising: a heavy chain variable domain comprising an amino acid sequence of SEQ ID NO:2, or an amino acid sequence at at least 90% identical to SEQ ID NO:2, and/or a light chain variable domain comprising an amino acid sequence of SEQ ID NO:7, or an amino acid sequence at least 90% identical to the SEQ ID NO:; and/or a monoclonal antibody against OmpW2 or Acinetobacter baumannii or an antigen-binding fragment thereof, comprising a heavy chain variable domain comprising an amino acid sequence of SEQ ID NO:12 or 22, or an amino acid sequence at least 90% identical to the SEQ ID NO:12 or 22, and/or a light chain variable domain comprising an amino acid sequence of SEQ ID NO:17 or 27, or an amino acid sequence at least 90% identical to the SEQ ID NO:17 or 27.

26. The composition of claim 25, wherein the composition is a pharmaceutical composition, comprising pharmaceutically acceptable carriers or excipients selected from preservatives, adjuvants, stabilizers, surfactants, amino acids, antioxidants, buffer, lyoprotectant, and other additives such as mannitol, BSA, blood serum, and skim milk, or combination thereof.

27. The composition of claim 25, wherein the composition can optionally further comprise at least one antibiotic against A. baumannii and/or other ESKAPEE pathogens.

28. The composition of claim 25, wherein the composition can optionally further comprise antibodies that can be polyclonal or monoclonal antibodies, targeting other epitopes of A. baumannii BauA and/or OmpW2, and/or other bacterial antigens of A. baumannii or other ESKAPEE pathogens.

29. The composition of claim 26, wherein the antibody is one selected from antibodies against hemolysin co-regulated protein (Hcp), fimbrial protein A chain (FimA), OmpW3, or combination thereof.

30. A method of administering the composition of claim 25 to a subject for prophylactic or therapeutic treatment of A. baumannii infection, wherein the composition is administered between one days prior to and 1-2 days after possible A. baumannii infection.

31. (canceled)

32. (canceled)

33. (canceled)

34. The method of claim 30, wherein the dose of the monoclonal antibody against BauA and/or OmpW2 of A. baumannii or an antigen-binding fragment thereof for parenteral route administration is 10-50 mg/kg bodyweight.

35. (canceled)

36. (canceled)

37. (canceled)

38. (canceled)

39. (canceled)

40. (canceled)

41. (canceled)

42. (canceled)

43. (canceled)

44. The monoclonal antibody against BauA of Acinetobacter baumannii or an antigen-binding fragment thereof of claim 1, wherein BauA epitope comprises the amino acid sequence of SEQ ID NO:31 (ADEPFA).

45. The monoclonal antibody against OmpW2 of Acinetobacter baumannii or an antigen-binding fragment thereof of claim 9, wherein OmpW2 epitope comprises the amino acid sequence SEQ ID NO:32 (QNVLDG)
